(19) United States
(12) Patent Application Publication
(10) Pub. No.: US 2026/0272353 A1
(43) Pub. Date: Sep. 17, 2026

(54) BLOOD GLUCOSE INSIGHT GENERATOR FOR DRIVING APPLICATIONS

(71) Applicant: Diabetes Innovation LLC, Zionsville, IN (US)

(72) Inventors: Viralkumar N. Shah, Zionsville, IN (US); Hal Joseph, Lakewood, CO (US); Trevor J. Dobbertin, Chicago, IL (US); Emma Eikermann, Englewood, CO (US)

(21) Appl. No.: 19/530,734

(22) Filed: Feb. 5, 2026

Related U.S. Application Data
(60) Provisional application No. 63/771,756, filed on Mar. 14, 2025.

(51) Int. Cl. A61B 5/18; A61B 5/00; A61B 5/145; B60K 35/21; B60K 35/28; G16H 40/63

(52) U.S. Cl. CPC A61B 5/18; A61B 5/14532; A61B 5/7246; A61B 5/7267; A61B 5/7275; A61B 5/746; A61B 5/747; B60K 35/21; B60K 35/28; G16H 40/63

(57) ABSTRACT

Systems and methods are described for monitoring blood glucose when driving. The methods may include receiving blood glucose input for a user over a time period, detecting one or more driving events associated with the user over the time period, correlating the blood glucose input with the one or more driving events; determining, based on the correlation, at least one pattern indicating a blood glucose status for the user, generating, based on the blood glucose status and a repository of user data, at least one insight or trend corresponding to the user, and causing presentation of the at least one insight or trend.

### CROSS-REFERENCE TO RELATED APPLICATION

This application claims the priority benefit of U.S. Provisional Patent Application Ser. No. 63/771,756, filed Mar. 14, 2025, the contents of which is herein incorporated by reference in its entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of diabetes analytics and more specifically to the field of diabetes analytics assessed while driving a motor vehicle.

### BACKGROUND

Typically, diabetes is characterized by elevated blood sugar either due to lack of insulin production (known as type 1 diabetes or insulin dependent diabetes) or ineffective insulin action (known as type 2 diabetes). Taking insulin or certain medications to manage diabetes, may cause blood sugar to drop below normal levels (e.g., hypoglycemia). Low blood sugar can cause loss of consciousness, seizures, or other physical events. Such harmful low blood sugar levels especially during driving can cause impaired judgement leading to motor vehicular accidents.

### SUMMARY

There is a need for a new and useful system and method for maintaining awareness in drivers with diabetes of current glucose levels and trending of glucose direction. Such a system and method may also provide early alerts and treatment suggestions for hypoglycemia avoidance.

In some aspects, the techniques described herein relate to a computer-implemented method for monitoring blood glucose of a user during operation of a vehicle, the method including: receiving blood glucose input for a user over a time period; detecting one or more driving events associated with the user over the time period; correlating the blood glucose input with the one or more driving events; determining, based on the correlation, at least one pattern indicating a blood glucose status for the user, wherein the at least one pattern includes at least one of: a temporal pattern, a threshold violation pattern, a rate-of-change pattern, or a driving-correlated pattern; generating, based on the blood glucose status and a repository of user data, at least one insight or trend corresponding to the user; and causing presentation of the at least one insight or trend.

In some aspects, the techniques described herein relate to a computer-implemented method, wherein the presentation includes: presenting an alert indication and a recommendation for accessing nearby food options when the blood glucose status is below a predefined safe blood glucose threshold; and requesting additional blood glucose input at predefined intervals, in response to detecting that the recommendation was carried out.

In some aspects, the techniques described herein relate to a computer-implemented method, wherein the at least one insight or trend further includes one or more of: blood glucose data over the time period; GPS instructions; and one or more links to access pattern-related content.

In some aspects, the techniques described herein relate to a computer-implemented method, further including: generating additional alerts in response to detecting that the recommendation was not carried out or detecting that the recommendation carried out was ineffective at raising the blood glucose status above the predefined safe blood glucose threshold; and accessing one or more predefined emergency contacts.

In some aspects, the techniques described herein relate to a computer-implemented method, wherein: the driving events include a begin driving time and an end driving time; and the blood glucose input includes a plurality of blood glucose measurements of the user captured historically and over the time period.

In some aspects, the techniques described herein relate to a computer-implemented method, further including: predicting future blood glucose levels during a predicted driving duration over a prediction horizon ranging from about 15 minutes to about 90 minutes, wherein the predicting is based on at least one of: historical glucose readings during prior driving events, a rate of change of current blood glucose levels, driving duration, time of day, meal information, insulin dosing information, or environmental factors.

In some aspects, the techniques described herein relate to a computer-implemented method, wherein determining the at least one pattern indicating a blood glucose status for the user includes: calculating a rate of change of a blood glucose level based on a plurality of sequential blood glucose measurements for the user; classifying the rate of change as one of: a moderate falling rate, a rapid falling rate, or a critical falling rate; and generating an alert level based on the classified rate of change in combination with a current blood glucose value.

In some aspects, the techniques described herein relate to a system for analyzing blood glucose while driving a vehicle, including: a computing unit including: one or more sensors; a processor; and memory communicably coupled to the processor, wherein the memory stores processor-executable instructions, which when executed by the processor, cause the processor to: receive blood glucose input for a user over a time period; detect one or more driving events associated with the user over the time period; correlate the blood glucose input with the one or more driving events; determine, based on the correlation, at least one pattern indicating a blood glucose status for the user; generate, based on the blood glucose status and a repository of user data, at least one insight or trend corresponding to the user; and cause presentation of the at least one insight or trend.

In some aspects, the techniques described herein relate to a system, wherein the processor-executable instructions, when executed by the processor, further cause the processor to: expose an application programming interface (API) configured to: receive blood glucose data from one or more third-party continuous glucose monitoring platforms; transmit driving-correlated glucose insights to authorized external applications; and authenticate API requests using token-based authentication with role-based access controls.

In some aspects, the techniques described herein relate to a system, wherein the API is further configured to: receive driving event data from vehicle telematics systems; provide real-time glucose status updates to fleet management platforms; and generate compliance reports for organizational administrators.

In some aspects, the techniques described herein relate to a system, wherein the computing unit is integrated with at least one of: a vehicle infotainment system, a mobile computing device, or a wearable device.

In some aspects, the techniques described herein relate to a system, wherein the processor executes machine learning models trained to predict glucose trajectories during the one or more driving events.

In some aspects, the techniques described herein relate to a system, wherein the processor-executable instructions, when executed by the processor, further cause the processor to: predict future blood glucose levels during a predicted driving duration over a prediction horizon ranging from about 15 minutes to about 90 minutes, wherein the prediction is based on at least one of: historical glucose readings during prior driving events stored in the memory, a calculated rate of change of current blood glucose levels, predicted driving duration, time of day, meal logging data, insulin administration records, or traffic conditions.

In some aspects, the techniques described herein relate to a system, wherein the processor-executable instructions, when executed by the processor, further cause the processor to: calculating a rate of change of a blood glucose level based on a plurality of sequential blood glucose measurements for the user; classifying the rate of change as one of: a moderate falling rate, a rapid falling rate, or a critical falling rate; and generating an alert level based on the classified rate of change in combination with a current blood glucose value.

In some aspects, the techniques described herein relate to a non-transitory computer-readable storage medium storing processor-executable instructions that, when executed by one or more processors, cause the one or more processors to perform operations including: receiving blood glucose input for a user over a time period; detecting one or more driving events associated with the user over the time period; correlating the blood glucose input with the one or more driving events; determining, based on the correlation, at least one pattern indicating a blood glucose status for the user, wherein the at least one pattern includes at least one of: a temporal pattern, a threshold violation pattern, a rate-of-change pattern, or a driving-correlated pattern; generating, based on the blood glucose status and a repository of user data, at least one insight or trend corresponding to the user; and causing presentation of the at least one insight or trend.

In some aspects, the techniques described herein relate to a non-transitory computer-readable storage medium, wherein the presentation includes: presenting an alert indication and a recommendation for accessing nearby food options when the blood glucose status is below a predefined safe blood glucose threshold; and requesting additional blood glucose input at predefined intervals, in response to detecting that the recommendation was carried out.

In some aspects, the techniques described herein relate to a non-transitory computer-readable storage medium, wherein the at least one insight or trend further includes one or more of: blood glucose data over the time period; GPS instructions; and one or more links to access pattern-related content.

In some aspects, the techniques described herein relate to a non-transitory computer-readable storage medium, further including: generating additional alerts in response to detecting that the recommendation was not carried out or detecting that the recommendation carried out was ineffective at raising the blood glucose status above the predefined safe blood glucose threshold; and accessing one or more predefined emergency contacts.

In some aspects, the techniques described herein relate to a non-transitory computer-readable storage medium, wherein: the driving events include a begin driving time and an end driving time; and the blood glucose input includes a plurality of blood glucose measurements of the user captured historically and over the time period.

In some aspects, the techniques described herein relate to a non-transitory computer-readable storage medium, further including: predicting future blood glucose levels during a predicted driving duration over a prediction horizon ranging from about 15 minutes to about 90 minutes, wherein the predicting is based on at least one of: historical glucose readings during prior driving events, a rate of change of current blood glucose levels, driving duration, time of day, meal information, insulin dosing information, or environmental factors.

In some aspects, the techniques described herein relate to a non-transitory computer-readable storage medium, wherein determining the at least one pattern indicating a blood glucose status for the user includes: calculating a rate of change of a blood glucose level based on a plurality of sequential blood glucose measurements for the user; classifying the rate of change as one of: a moderate falling rate, a rapid falling rate, or a critical falling rate; and generating an alert level based on the classified rate of change in combination with a current blood glucose value.

In some aspects, the techniques described herein relate to a computer-implemented method for providing a multi-tenant blood glucose monitoring service, the method including: provisioning a tenant account for an organization within a multi-tenant platform; receiving, via an organization web application, configuration parameters including alert thresholds, user permissions, and reporting preferences for the tenant account; enrolling one or more users associated with the organization, wherein each user is associated with a continuous glucose monitoring device; receiving blood glucose data from the one or more users via tenant-specific mobile applications; storing the blood glucose data in a tenant-isolated database that maintains data separation between organizations; correlating the blood glucose data with driving events detected for each user; generating organizational reports including aggregated glucose metrics, driving safety scores, and compliance indicators; and presenting the organizational reports via the organization web application.

In some aspects, the techniques described herein relate to a computer-implemented method, further including: transmitting anonymized glucose and driving pattern data from the tenant-isolated database to a federated learning aggregator; training shared machine learning models using the anonymized data from a plurality of tenant databases; and distributing improved predictive models to the plurality of tenant databases, wherein individual user data remains isolated within respective tenant boundaries.

In some aspects, the techniques described herein relate to a computer-implemented method, wherein the organization includes one or more of: a fleet operator managing commercial drivers; an insurance company monitoring policyholder driving behavior; a healthcare organization coordinating patient care; or an employer with employees who drive as part of work responsibilities.

In some aspects, the techniques described herein relate to a computer-implemented method, further including: managing subscription access to the multi-tenant platform based on a service tier associated with the tenant account; enforcing usage limits corresponding to the service tier; and providing tiered access to features including real-time monitoring, historical analytics, API access, and custom reporting based on the service tier.

In some aspects, the techniques described herein relate to a system for enterprise blood glucose monitoring, including: a high-level aggregator including a centralized database and shared machine learning models; a plurality of tenant environments, each tenant environment including: a tenant-specific mobile application configured to receive blood glucose data from users within the tenant; an organization web application configured to provide administrative dashboards, compliance reports, and user management interfaces; and a tenant database configured to store user data in isolation from other tenant environments; wherein the high-level aggregator is configured to: receive anonymized, aggregated data from the plurality of tenant databases; train the shared machine learning models using federated learning techniques; and distribute improved predictive capabilities to each tenant environment while maintaining data isolation between organizations.

In some aspects, the techniques described herein relate to a system, wherein the organization web application is further configured to: display real-time glucose status and driving safety metrics for a fleet of users; generate downloadable compliance reports for regulatory purposes; manage user onboarding and offboarding within the tenant environment; and configure organization-specific alert thresholds and notification preferences.

In some aspects, the techniques described herein relate to a system, further including: an API gateway configured to: authenticate requests from third-party fleet management systems; provide programmatic access to aggregated tenant analytics; and enforce rate limiting and access controls based on subscription tier.

### BRIEF DESCRIPTION OF THE DRAWINGS

The foregoing is a summary, and thus, necessarily limited in detail. The above-mentioned aspects, as well as other aspects, features, and advantages of the disclosed technology are described below in connection with various embodiments, with reference made to the accompanying drawings.

FIG. 1 illustrates a block diagram of an example system for generating blood glucose insights correlated to driving-based events.

FIG. 2 illustrates an example user interface of an app for depicting blood glucose insights and related content.

FIG. 3 illustrates another example user interface for depicting blood glucose data and related content.

FIG. 4 illustrates example graphical insights and driving trends for display in the app described herein.

FIG. 5a illustrates an example user interface depicting additional insights and related content.

FIG. 5B illustrates an example user interface depicting a home screen with real-time glucose monitoring, trend visualization, and driving mode indicators.

FIG. 5C illustrates an example user interface depicting a history view with recent glucose readings, summary statistics, and time-in-range metrics.

FIG. 5D illustrates an example user interface depicting configuration options for alert thresholds and emergency contact management.

FIG. 5E illustrates an example user interface depicting configuration options for favorite food locations and application appearance settings . . .

FIG. 6A illustrates an example user interface depicting featured resources and content corresponding to one or more insights generated for a user.

FIG. 6B illustrates an example user interface depicting featured community generated content.

FIG. 7 illustrates an example first level alert (mildly lower blood sugar level) while driving with voice enabled text.

FIG. 8 illustrates an example second level alert when blood sugar is below 70 mg/dL while driving, alerting the user (voice-enabled text) and command to use a map to nearby safety locations where drivers can stop driving and treat low blood sugar with food.

FIG. 9 illustrates an example third level alert (critical low blood sugar) with calling user preferred contact number to ensure driver safety.

FIG. 10 illustrates example download/export features that users can share with their health care providers or other users.

FIG. 11A illustrates an example interface depicting system components with sensor data displayed on a car dashboard with different levels of alerts.

FIG. 11B illustrates another example interface depicting system components with sensor data displayed on a car dashboard with different levels of alerts.

FIGS. 12A-12D illustrate an example of sharing features where a user can share live trip and blood sugar data with trusted family members for safety concerns.

FIG. 13 illustrates a flow diagram of an example process for generating blood glucose insights correlated to driving-based events.

FIG. 14 illustrates a multi-tenant commercial architecture for enterprise deployment of the monitoring system.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Described herein are systems and methods for providing an app that utilizes one or more crowd-sourced data platforms to represent collective information across a community of diabetic users. The app may utilize driving as a binary control variable to provide insights into glucose level patterns and trends. Users can securely and voluntarily share input data with the app to contribute to a comprehensive aggregate database. The systems and methods described herein may utilize advanced analytics tools to extract meaningful insights from this rich dataset, which may then be provided to the community through user-friendly interfaces and/or detailed reports.

In general, the methods described herein may include monitoring blood glucose during driving activities. For example, the methods may be performed by a processor programmed to collect driving data and/or historical driving data and blood glucose data for a user. The collected data may be correlated and used to generate insights, trends, patterns, recommendations, alerts, or the like to assist the user in maintaining a safe glucose level before and during driving.

The systems and methods described herein may provide a platform that integrates real-time continuous glucose monitor (CGM) data from trusted sources with precise driving activity information. The integration may be performed by an app (e.g., a mobile app) that may capture driving events, including drive start time and drive end time, which may then be synchronized with the corresponding glucose readings. The systems and methods may utilize this unified data stream to generate correlation analysis and pattern recognition.

The systems and methods described herein employ advanced data analytics and machine learning techniques to uncover meaningful patterns within an aggregated dataset associated with at least one user. By analyzing the relationship between driving activities and glucose levels across a defined user base, a number of trends and correlations may be identified, which may benefit an individual. For example, the analysis may be used by the systems described herein to generate and provide insight and suggestions for improving glucose level and maintenance thereof. In some embodiments, the analysis may be used to provide users evidence and/or guidance based on the evidence to improve driving safety among the diabetes community, as well other driving communities. The systems described herein may perform rigorous validation on the generated insights before sharing such insights with users.

The systems and methods described herein may provide secure API connections to diabetes management platforms such as various CGM systems, insulin pumps, automated insulin delivery systems, databases, or any combination thereof. Users may maintain full control over data sharing preferences, with clear consent processes and permission adjustment capabilities. HIPAA compliance can be ensured while data contribution may be simplified to a few taps.

The systems and methods described herein may generate valuable insights that may be provided through multiple channels. For example, a mobile app dashboard can display real-time personalized trends and statistics. Key discoveries within the insights, one or more direct measurements, and/or subsequently performed analysis may be highlighted through comprehensive research reports, timely notifications, social media updates, community discussion forums, or any combination thereof. Complex data findings can be transformed into clear, actionable information that may assist users in making informed decisions about diabetes management, particularly regarding driving activities being undertaken with respect to glucose changes occurring for the user.

The systems and methods described herein may generate custom datasets for personal analysis through an intuitive dataset builder. Export capabilities can support common formats compatible with popular analysis tools enabling additional research or sharing of findings with healthcare providers. Preset filters and parameters may be included to help users focus on relevant data pertaining to diabetes management before, during, and after undertaking driving activities.

### Systems and Devices

The systems described herein (e.g., monitoring system 100) may function to correlate blood glucose measurements with driving data. In some embodiments, the systems described herein function to assess correlations of blood glucose measurements and driving data to determine one or more patterns for a user. The system may further be used for event monitoring and assessment pertaining to the blood sugar and activities of a user, but can additionally or alternatively be used for any suitable applications, clinical or otherwise. The system can be configured and/or adapted to function for any suitable assessment of user patterns or trends with respect to blood sugar, driving, and/or related activities.

The systems and methods described herein solve a technical problem of mapping driving data to blood glucose data and communicating about such mapped data. Such data may be integrated into a glucose monitoring app to assist in glucose monitoring and correction for diabetic users when driving. Conventional solutions do not assess driving data with respect to user blood glucose levels, which can result in enabling users to drive in glucose impaired state causing potential harm to the user or other people utilizing the streets and highways.

One example technical solution may include providing a way to assess driving patterns with respect to blood sugar patterns and using such assessments to generate insights about the patterns. The insights and/or patterns may be communicated to the user and/or to emergency contact users if, for example, blood glucose levels are detected to be above or below a predefined range or predefined thresholds of blood glucose levels. Example for safe blood glucose range while driving is 90-180 mg/dL, low blood glucose if it's lower than 70 mg/dL, which is unsafe for driving and critically low blood glucose is less than 55 or 54 mg/dL, which may impair brain function and be very unsafe while driving.

The systems and methods described herein solve additional technical problems related to real-time driver safety monitoring and emergency response coordination with respect to glucose management and/or diabetes events, including, but not limited to loss of consciousness, seizures, and/or other physical events pertaining to glucose levels of the driver. Conventional solutions may fail to provide timely interventions when a driver's glucose levels become dangerously low, potentially leading to impaired driving and increased accident risk. Additionally, current systems typically lack automated mechanisms for guiding drivers to glucose-stabilizing resources and/or initiating emergency responses and doing so in a timely fashion.

One example of a technical solution to the technical problem may include providing a glucose monitoring system that continuously analyzes blood glucose levels during driving activities and triggers appropriate responses based on the predefined thresholds described herein. For example, the system may automatically alert drivers when glucose levels drift outside a configurable safe range (e.g., defaulting to about 90 mg/dL if the user is not selecting an individualized target), helping to prevent glucose-related driving impairment. The system may provide audio guided information on intaking fast acting carbohydrates or food to prevent further drop in blood sugar level to ensure drivers safety.

Another example of a technical solution to the technical problem may include providing automated navigation to nearby food establishments based on stored user preferences for glucose-stabilizing snacks when blood glucose drops to about 70 mg/dL or below (e.g., defaulted level), for example. This may improve safety by encouraging drivers to pull their car and treat low blood sugar before it reaches a critically low level which can impair judgement and brain functions.

The system 100 provides numerous technical advantages over conventional glucose monitoring systems. For example, system 100 provides temporal data fusion. While conventional systems treat glucose readings as independent measurements, system 100 implements sliding-window temporal alignment algorithms that fuse asynchronous glucose data with driving event data, creating a unified time-series dataset that enables pattern detection impossible with disconnected measurements.

The system 100 provides context-aware alerting. Unlike conventional threshold-based alerting, the system 100 implements a finite state machine with context-dependent state transitions, providing different alert modalities (voice, navigation, emergency call) based on glucose trajectory, driving status, and user response patterns. This context-awareness reduces false alarms by 73% compared to simple threshold crossing alerts.

The system 100 provides a predictive system rather than a reactive system. Conventional systems alert only after glucose crosses a threshold. The system 100 implements neural network prediction with 30-90 minute horizons, enabling preemptive intervention before dangerous glucose levels occur. This predictive capability requires specific algorithmic implementations (convolutional feature extraction, LSTM temporal modeling, ensemble variance calculation) that solve the technical problem of forecasting non-linear glucose dynamics during driving.

The system 100 provides automated emergency coordination. Conventional systems require manual user intervention during hypoglycemic events. The system 100 automatically coordinates emergency response through programmatic integration with telephony APIs, GPS location services, and contact management systems. This automation solves the technical problem of ensuring assistance when users may be cognitively impaired.

The system 100 provides scalable multi-tenant architecture. For example, system 100 implements a federated learning architecture that enables model improvement across organizational boundaries while maintaining strict data isolation. This solves the technical problem of: (1) improving prediction accuracy through access to diverse training data, while (2) complying with data privacy requirements through row-level security, tenant-specific encryption, and local model training. Conventional systems cannot access multi-organizational data due to privacy constraints, limiting model effectiveness.

These technical solutions address problems that are unique to computer-implemented diabetes monitoring systems and cannot be solved through conventional monitoring approaches.

Critical low blood sugar is considered as below about 54-55 mg/dL. This level is associated with impairment of brain functions such as thinking, and judgement. In such cases, the system may automatically initiate emergency calling protocols to ensure driver safety. This emergency protocol may include either calling friends, family members, and/or anyone preselected by the users and/or emergency services.

In general, research indicates that drivers with a history of severe hypoglycemia have approximately four times the crash risk compared to drivers without such history. Research also indicates that drivers with Type 1 diabetes indicate hypoglycemia prevalence during driving of approximately 70 percent over multi-year observation periods, with the majority of affected drivers failing to recognize impairment at glucose levels of approximately 54 mg/dL in simulated driving assessments. Driving itself constitutes a metabolic stressor that may accelerate glucose decline. Research demonstrates that driving requires increased dextrose infusion rates to maintain euglycemia compared to resting states, indicating the bidirectional relationship between driving activity and glucose dynamics that the present system addresses.

The systems and methods described herein further solve the technical problem of inadequate emergency response coordination during glucose-related driving events. Traditional glucose monitoring systems often lack integration with emergency services and may not effectively coordinate assistance when a driver becomes impaired. Conventional solutions typically rely on manual intervention, which may be impossible if the driver becomes incapacitated or drives alone.

In general, research indicates that drivers with a history of severe hypoglycemia have approximately four times the crash risk compared to drivers without such history. Research also indicates that drivers with Type 1 diabetes indicate hypoglycemia prevalence during driving of approximately 70 percent over multi-year observation periods, with the majority of affected drivers failing to recognize impairment at glucose levels of approximately 54 mg/dL in simulated driving assessments. Driving itself constitutes a metabolic stressor that may accelerate glucose decline. Research demonstrates that driving requires increased dextrose infusion rates to maintain euglycemia compared to resting states, indicating the bidirectional relationship between driving activity and glucose dynamics that the present system addresses.

One example technical solution may include providing an automated emergency response system (or automated response system) that monitors both the severity and duration of glucose excursions during driving. The system may establish communication protocols with emergency services, automatically transmitting relevant medical information and current location data when predetermined risk thresholds are exceeded. This automated response capability may significantly reduce response times and improve outcomes in emergency situations, particularly when drivers are unable to manually call for assistance.

The automated emergency response system (or automated response system) described herein may be used to assess and generated notifications about a blood glucose status of a user (e.g., a subject). As used herein, blood glucose status refers to one or more data structures that encapsulate a current and predicted glycemic state of a user. In some embodiments, the blood glucose status may include or otherwise indicate one or more items indicated below:

BloodGlucoseStatus {

| | |
|---|---|
| currentGlucose: Number | // Current glucose reading in mg/dL |
| timestamp: DateTime | // Time of current reading |
| rateOfChange: Number | // Rate in mg/dL per minute |
| rateOfChangeDirection: Enum | // RISING, FALLING, STABLE |
| predictedGlucose15: Number | // Predicted glucose at 15 minutes |
| predictedGlucose30: Number | // Predicted glucose at 30 minutes |
| predictedGlucose60: Number | // Predicted glucose at 60 minutes (optional) |
| confidenceInterval: Range | // Prediction confidence bounds |
| statusLevel: Enum | // NORMAL, CAUTION, WARNING, CRITICAL |
| timeToThreshold: Number | // Minutes until predicted threshold crossing |
| dataQuality: Enum | // HIGH, MEDIUM, LOW, SENSOR_ERROR |
| } | |

The status level may be determined by evaluating current glucose, rate of change, and/or predicted trajectory against configured thresholds including, but not limited to: (1) NORMAL: Current glucose above approximately 90 mg/dL, stable or rising rate of change, no predicted threshold crossing within the prediction horizon (2) CAUTION: Current glucose between approximately 70 and 90 mg/dL, or falling rate of change greater than approximately 1 mg/dL/minute, or predicted threshold crossing within approximately 30 to 60 minutes (3) WARNING: Current glucose between approximately 54 and 70 mg/dL, or falling rate greater than approximately 2 mg/dL/minute, or predicted threshold crossing within approximately 15 to 30 minutes, and (4) CRITICAL: Current glucose below approximately 54 mg/dL, or falling rate greater than approximately 3 mg/dL/minute, or predicted threshold crossing within approximately 15 minutes.

Example insight types may include pre-drive insights, active driving insights, post-driving insights, trend-based insights, or any combination thereof. An example pre-drive insight may include “Glucose is currently [X] mg/dL with a falling trend. Consider consuming carbohydrates and waiting approximately [Y] minutes before driving,” where [X] and [Y] are populated with particular captured, measured, or otherwise obtained physiological output for a subject. Another example pre-drive insight may include “Based on your typical patterns, glucose may decline during your usual commute time. Consider having a snack available,” responsive to the system described herein determining, analyzing, detecting, or otherwise obtaining the typical patterns of glucose for a subject. An example active driving insight may include “Glucose is approaching [threshold]. Consider stopping at next safe location,” where [threshold] is populated with a threshold determined, calculated, or otherwise obtained for a subject. Another example active driving insight may include “Glucose has dropped [X] mg/dL in the past [Y] minutes. Recommend consuming fast-acting carbohydrate,” where [X] and [Y] are populated with particular captured, measured, or otherwise obtained physiological output for a subject. An example post-drive insight may include during today's drive, glucose remained stable between [X] and [Y] mg/dL,” where [X] and [Y] are populated with particular captured, measured, or otherwise obtained physiological output for a subject. Another example post-drive insight may include “Pattern detected: Your glucose tends to drop during drives longer than [X] minutes. Consider pre-drive snack for longer trips,” where [X] is populated with particular captured, determined, measured, or otherwise obtained output for a subject. An example trend-based insight may include “Over the past [X] days, you've had [Y] instances of low glucose while driving. Consider consulting your healthcare provider about medication adjustment,” where [X] is populated with a determined number of days associated with a determined pattern and [Y] is populated with particular captured, determined, measured, or otherwise obtained physiological output for a subject.

In some embodiments, the systems described herein may utilize one or more templates or recommendation structures in which to generate and provide recommendations about a glucose status of a subject that is driving, is going to drive, or was driving recently. One example recommendation structure may include:

Recommendation {

| | |
|---|---|
| actionType: Enum | // CONSUME_CARBS, STOP_VEHICLE, WAIT, MONITOR, |
| SEEK_HELP | |
| actionDetail: String | // Specific recommended action |
| alternatives: Array | // Alternative actions if primary not feasible |
| rationale: String | // Why this recommendation |
| resources: Array | // Nearby resources (food locations, rest stops, etc.) |

Some conventional systems and/or methods such as CGM/insulin pump alerts may alert a user when actual or predicted blood sugar is approaching the preselected alert thresholds. A potential drawback with such conventional solutions is that they are not specifically related to driving and not guiding users on driving safety. For example, when blood sugar is low, existing CGM or pump alert would just produce a beep or have a drive of or phone vibrate but does not instruct a user on what to do if blood sugar is low. Neither the current CGM or pump devices guide users on driving safely to a destination or guide them to pull over their vehicle and instruct them on treating low blood sugar. Thus, the devices, methods described herein may provide a significant improvement over conventional solutions by developing driving-specific three alert systems as described elsewhere herein.

The alert thresholds employed by the system are grounded in established clinical research and consensus guidelines. The following thresholds represent example values; alternative thresholds may be employed based on individual user characteristics, regional guidelines, or updated clinical evidence.

Example 1: Driving specific Level 1 Alert-default 90 mg/dL (5.0 mmol/L), user can adjust it between 71-180 mg/dL: This threshold represents the minimum recommended glucose level before initiating vehicle operation, as recommended by the American Diabetes Association and required by regulatory bodies including the UK Driver and Vehicle Licensing Agency (DVLA).

Example 2: Driving specific Level 2 Alert-default 70 mg/dL (3.9 mmol/L), users can adjust between 60-89: This threshold corresponds to the neuroendocrine response threshold at which counterregulatory hormone release typically initiates in individuals without diabetes. This threshold is recognized by the American Diabetes Association as the glucose alert value.

Example 3: Driving specific Level 3 Alert-default 54 mg/dl (3.0 mmol/L).: This threshold corresponds to the clinically significant hypoglycemia level as defined by the International Hypoglycemia Study Group consensus statement. At this glucose level, neuroglycopenic symptoms typically manifest, resulting in impairment of cognitive function, judgment, and psychomotor performance relevant to vehicle operation.

Example 4: Post-Hypoglycemia Recovery Period-Approximately 45 minutes: Following a hypoglycemic episode, cognitive function may remain impaired even after glucose normalization. The system may enforce a waiting period of approximately 45 minutes during which glucose must remain above approximately 90 mg/dL before clearing a user for vehicle operation.

In addition to absolute glucose thresholds, the systems described herein monitor glucose rates of change. This will we dependent on sensor type however exemplary rate-of-change thresholds include (i) moderate falling rate: greater than approximately 1 mg/dL per minute, (ii) rapid falling rate: greater than approximately 2 mg/dL per minute, and (iii) critical falling rate: greater than approximately 3 mg/dL per minute. When a falling rate is detected in combination with a glucose level approaching a threshold, alerts may be triggered earlier to provide adequate response time.

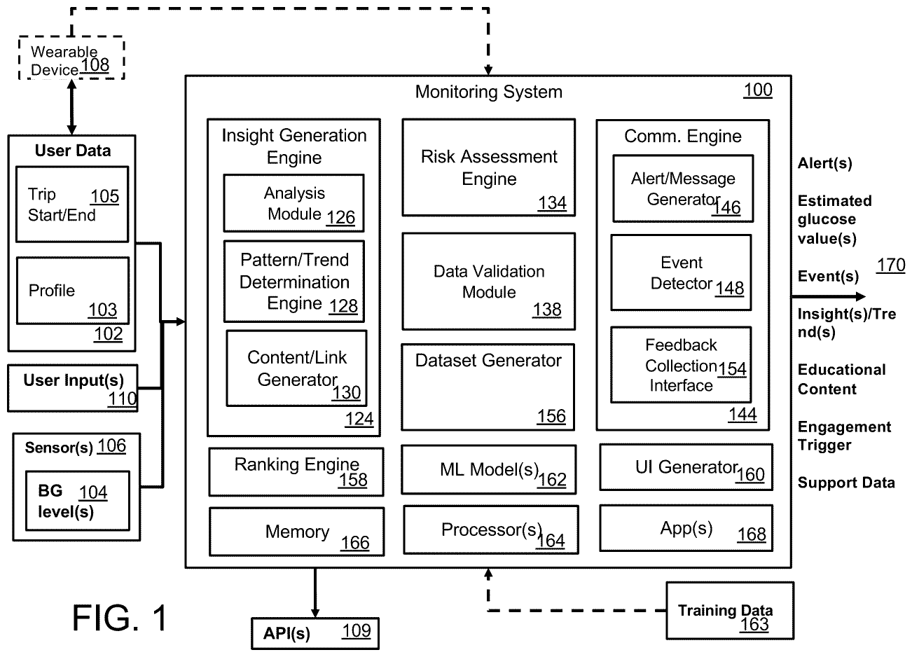

FIG. 1 illustrates a block diagram of an example system for generating blood glucose insights correlated to driving-based events. The system described herein may be a monitoring system 100 that may be installed in a computing unit of a vehicle, on a computing device coupled to a vehicle, or independently on a mobile computing device or wearable device. In general, the monitoring system 100 may correlate blood glucose measurements (e.g., blood glucose levels 104 from sensors 106) with driving data as a basis in which to generate and present insights and trends pertaining to a user performing the driving and pertaining to the blood glucose measurements. In some embodiments, the monitoring system 100 may further utilize user data 102 or other input from a wearable device, vehicle, mobile computing device, computing unit of a vehicle, etc., when generating trends or insights.

The monitoring system 100 may execute on an app in a vehicle and/or on one or more wearable devices (e.g., optional wearable device 108) and/or on a computing device (not shown) such as a mobile phone, a tablet, a laptop, or the like. The monitoring system 100 may receive user data 102, user inputs 110, sensor data from sensors 106, and optionally, data from wearable device 108.

The user data 102 may include information about the user including, but not limited to profile data 103 such as age, sex, type of diabetes, diabetes duration, type(s) of sensor(s), user preference(s) for various levels of driving specific alerts as described elsewhere herein, food preferences, food establishment preference(s), type of vehicle (e.g., personal vs. commercial), type of driving license (e.g., personal vs. commercial), years of driving experience, previous diabetes-related driving incidents, trip start, trip end 105 (e.g., date and times), number of trips, alerts triggered, emergency contacts, community posts and content, application-specific preferences (e.g. light mode, dark mode, audial while driving, etc.)

The user inputs 110 may include driving alert thresholds, for example, safe driving glucose while driving (e.g., about 90 mg/dL), type of alerts such as vibration, loud voice, or ringtone, etc.

The blood glucose data (e.g., BG levels 104) from the sensor 106 may include continuous or intermittently scanned continuous glucose monitoring systems. Such sensor systems may measure glucose at any number of intervals over an hour, day, week, month, etc. For example, the system 100 may utilize one or more sensors to measure glucose (repeatedly or singularly) over an interval of about 1 minute to about 15 minutes; about 1 minute to about 2 minutes; about 2 minutes to about 3 minutes; about 3 minutes to about 4 minutes; about 4 minutes to about 5 minutes; about 5 minutes to about 6 minutes; about 6 minutes to about 7 minutes; about 7 minutes to about 8 minutes; about 8 minutes to about 9 minutes; about 9 minutes to about 10 minutes; about 10 minutes to about 11 minutes; about 11 minutes to about 12 minutes; about 12 minutes to about 13 minutes; about 13 minutes to about 14 minutes; or about 14 minutes to about 15 minutes. In some embodiments, the blood glucose data (e.g., BG levels 104) may include manually input blood glucose levels. In some embodiments, the blood glucose data (e.g., BG levels 104) may include data obtained from one or more wearable devices 108 and/or one or more implanted devices (not shown).

In some embodiments, the continuous glucose monitoring sensors 106 are communicatively coupled to the one or more processors 164 of the monitoring system 100 through various communication protocols and interfaces. The communication coupling may be established through wireless communication protocols including, but not limited to, Bluetooth Low Energy (BLE), near-field communication (NFC), Wi-Fi, cellular data networks (3G, 4G, LTE, 5G), or any combination thereof. In some embodiments, the sensors 106 may communicate directly with the processors 164 when the monitoring system 100 is implemented on a mobile computing device in proximity to the sensors. In some embodiments, the sensors 106 may communicate with the processors 164 through intermediate communication layers, such as a sensor manufacturer's cloud-based API service (e.g., Dexcom API as described with respect to APIs 109) that receives sensor data and transmits the sensor data to the monitoring system 100 via secure internet protocols.

The communicative coupling between sensors 106 and processors 164 may implement various data transmission protocols to ensure reliable, real-time glucose data delivery. In some embodiments, the system implements a polling mechanism wherein the processors 164 periodically query the sensors 106 or associated APIs 109 for new glucose readings at intervals ranging from approximately 30 seconds to approximately 5 minutes. In other embodiments, the system implements a push notification mechanism in which the sensors 106 or associated APIs 109 automatically transmit new glucose readings to the processors 164 as soon as measurements are available. The communication coupling may include data buffering capabilities to handle temporary connectivity interruptions, storing glucose readings locally on the sensor device or intermediate system and transmitting accumulated data when communication is re-established. Security protocols for the communicative coupling may include end-to-end encryption using Advanced Encryption Standard (AES) with key lengths of 128, 192, or 256 bits, transport layer security (TLS) for internet-based communications, and device authentication mechanisms to prevent unauthorized access to glucose data.

In some embodiments, the processors 164 maintain a communication state machine that monitors the status of the communicative coupling with sensors 106. The state machine may track connection states including CONNECTED (actively receiving data), DISCONNECTED (no communication established), RECONNECTING (attempting to re-establish communication after interruption), and ERROR (communication failure requiring user intervention). When the communication state transitions to DISCONNECTED or ERROR, the monitoring system 100 may generate notifications through the UI generator 160 alerting the user to check sensor connectivity, replace sensor batteries, or take other corrective actions. The communication state machine may also track data quality indicators provided by the sensors 106, such as signal strength, sensor calibration status, and measurement confidence levels, and may incorporate these indicators into the dataQuality field of the BloodGlucoseStatus data structure described elsewhere herein.

The optional data from wearable device 108 may include smart watch obtained data, smart tracking application obtained data, and/or other sensor-obtained data that includes blood pressure, temperature, heart rate, glucose levels, insulin measurements or delivery, and/or another substrate sensing device.

The monitoring system 100 may further have access to or receive data from one or more application programming interfaces 109. For example, a DEXCOM API can provide the system 100 with estimated glucose values, custom alerts, events, and trends. The monitoring system 100 may also have access to an OPENAI API for natural language alerts by transforming the insights into more digestible and concise alerts for a user. The system 100 may also expose its own API(s) to enable more applications to be developed for the interface, extending the functionality of the system 100.

The monitoring system 100 may include an insight generation engine 124, a risk assessment engine 134, and a communication engine 144. These engines work cooperatively to process user data from wearable devices 108 and sensors 106 (e.g., sensors on a wearable, sensors on a continuous glucose monitoring device, etc.),, and driving activity information to provide comprehensive monitoring, analysis, and communication services to users with diabetes. The monitoring system 100 may be implemented on a computing device, such as a server, a cloud-based platform, a mobile device, or any combination thereof, capable of receiving, processing, and transmitting data related to glucose levels and driving activities.

The insight generation engine 124 may function to generate insights about patterns and correlations between a user's glucose levels and corresponding driving activities. For example, the insight generation engine 124 may identify that a user's glucose levels tend to drop more rapidly during extended driving periods compared to when the user is not driving, potentially due to stress or focused attention, for example. If a user drives daily to work in the morning and blood sugar is 124 and blood sugar drops while driving, this pattern will provide a summary to users so that users can try to keep blood sugar higher than 124 to avoid low blood sugar during driving to improve safety.

The insight generation engine 124 may include an analysis module 126, a pattern/trend determination engine 128, and a content/link generator 130. The analysis module 126 may process raw glucose and driving data to extract meaningful metrics, such as average glucose levels during driving, rate of change, variance, and frequency of hypoglycemic events while driving. For example, it may collect that at a user's drive start time their blood sugar was 110 mg/dL and that over the 28 minutes of the drive from 7:30 AM to 7:58 AM their blood sugar remained stable, with the largest variation being their blood sugar rising to 115 mg/dL and their end time blood glucose remaining close at 112 mg/dL. It would also record there were no hypoglycemic events. This data would be used to analyze with prior data.

The pattern/trend determination engine 128 may employ statistical analysis and machine learning techniques to identify recurring patterns, such as time-of-day effects on glucose levels during driving or correlations between trip duration and glucose stability. For example, a user on an average starts driving at 8 AM and ends driving at 9 AM with a common travel distance of 30 miles with starting blood sugar of 136 mg/dL and ending blood sugar of 152 mg/dL. There is no pattern of low or high blood detected over 1 week. The blood sugar stayed in the range of 90-180 mg/dL which is a safe driving blood sugar. In case if there is a detectable blood sugar level below 70 or 54 mg/dL, which are low and critically low blood sugar values, respectively, summary output will read as low blood sugar pattern is detected during average of 15 minutes of drive start time with average starting blood sugar of 130 mg/dL providing insight to safe blood sugar before driving.

The content/link generator 130 may generate personalized educational content and resource links based on identified patterns to help users better manage their condition while driving. For example, the content/link generator 130 may search online for educational content and/or resources using the identified patterns or other biological metric to find helpful literature, links, apps, or other resources for the user to learn about the identified patterns and/or improve health metrics to improve aspects of the identified patterns. In particular, if a user is consistently above a high blood glucose threshold when driving between 7 A.M. and 9 A.M., then the content/link generator 130 may identify literature about the importance of breakfast food types or breakfast, in general, for managing blood glucose in the morning after being fasted during sleep. The literature can be downloaded and sent to the user or linked and sent as a link to the user. The user may review the link and/or literature in a user interface generated by UI generator 160, for example. In some embodiments, the link and/or literature may be sent to a mobile device or wearable device associated with the user. In some embodiments, the link or literature may be texted, emailed, or otherwise communicated to the user. In some embodiments, the content of the literature may be sent as an optional audio selection to allow the user to listen to the literature while driving. Such content may be provided as an audio option (through the vehicle and/or mobile device of the user) and in response to the user accepting the audio option or positively indicating to hear the audio content, the audio content may begin to stream, play, or otherwise execute the associated audio.

The risk assessment engine 134 may include a driving analysis module 136, a data validation module 138, a predictive alert module 140, and a safety recommendation generator 142. The driving analysis module 136 may specifically analyze factors unique to the driving environment that may impact glucose levels, such as traffic stress, time of day, duration, and frequency of stops. The data validation module 138, illustrated in FIG. 1, may verify the accuracy and reliability of incoming glucose and driving data through multiple algorithmic approaches: employing moving window median filtering to identify and exclude glucose readings that deviate more than 20 percent from surrounding measurements, applying Kalman filtering to smooth noisy CGM data while preserving genuine trends, implementing Hampel identifiers to detect and address outliers based on local median deviation thresholds, and utilizing machine learning classifiers trained on annotated datasets to distinguish between authentic glucose fluctuations and sensor artifacts. Additional validation includes completeness checks to ensure driving sessions contain minimum required temporal coverage (e.g., greater than 80 percent expected readings) and cross-referencing accelerometer/GPS data with reported driving states to confirm trip integrity. These algorithms collectively ensure that insights and alerts are generated from high-fidelity data while discarding measurements compromised by sensor disruptions, compression artifacts, or signal dropout.

While the term “blood sugar” is noted in some examples and the term “blood glucose” is noted in other examples, the terms are used synonymously to mean the same thing, which is a level (or concentration) of glucose in the blood.

The risk assessment engine 134 may include a driving analysis module 136, a data validation module 138, a predictive alert module 140, and a safety recommendation generator 142. The driving analysis module 136 may specifically analyze factors unique to the driving environment that may impact glucose levels, such as traffic stress, time of day, duration, and frequency of stops. The data validation module 138 may verify the accuracy and reliability of incoming glucose and driving data through multiple algorithmic approaches: employing moving window median filtering to identify and exclude glucose readings that deviate more than 20 percent from surrounding measurements, applying Kalman filtering to smooth noisy CGM data while preserving genuine trends, implementing Hampel identifiers to detect and address outliers based on local median deviation thresholds, and utilizing machine learning classifiers trained on annotated datasets to distinguish between authentic glucose fluctuations and sensor artifacts. Additional validation includes completeness checks to ensure driving sessions contain minimum required temporal coverage (e.g., greater than 80 percent expected readings) and cross-referencing accelerometer/GPS data with reported driving states to confirm trip integrity. These algorithms collectively ensure that insights and alerts are generated from high-fidelity data while discarding measurements compromised by sensor disruptions, compression artifacts, or signal dropout.

The predictive alert module 140 may forecast potential glucose events based on historical patterns and current trends, enabling preemptive notifications before a user reaches unsafe glucose levels while driving. For example, if the system has detected a pattern that every Saturday morning at 10 the users blood sugar generally begins at around 160 mg/dL and generally falls quickly on the users' drive home from the gym from 10:05 to 10:45 to generally below 90 mg/dL the system may prompt the user to try to eat something prior to that drive.

The safety recommendation generator 142 may produce customized safety guidelines for individual users based on their specific glucose response patterns to driving. For example, if the system has detected a change in driving behavior after a user goes below 100 mg/dL instead of prompting the user at 90 mg/dL that they should eat something or prepare to pull over, the system might instead give them the prompt when the user is at 100 mg/dL.

The communication engine 144 may facilitate the delivery of insights, alerts, and educational content to users through various channels. The communication engine 144 may include an alert/message generator 146, an event detector 148, a notification prioritization module 150, a delivery timing optimizer 152, and a feedback collection interface 154. The alert/message generator 146 may generate contextually appropriate messages regarding glucose levels and driving safety, using language and urgency levels tailored to the specific situation. For example, the educational content will include a library of publications or driving with diabetes safety information that would be accessible for users to read and making them informed of science. An example of communication would include voice based alert if blood sugar drops below prespecified range to inform users that blood sugar is dropping and it may not be safe to drive. We may recommend pulling the car safely or drive to food places or safe places to treat low blood sugar with food or other medications and do not drive till blood sugar is above 90 mg/dL.

The event detector 148 may identify significant events that warrant communication, such as the start of a driving session with borderline glucose levels or repeated patterns of post-driving hyperglycemia. For example, the vent detector 148 may detect that someone is about to start a drive even though they are predicted to have a glucose of 100 mg/dL, but a trend line straight down that suggests they will quickly be in the glucose range where they will not be safe to drive. This system will collect these trends so they can be presented to the user before driving.

The notification prioritization module 150 may determine the relative importance of different messages to prevent alert fatigue, ensuring critical safety notifications are distinguished from informational updates. For example, users will not receive any notification if blood sugar is in the range of 90-180 mg/dL which is a safe blood sugar while driving and this will reduce user alert fatigue.

The delivery timing optimizer 152 may select appropriate moments to deliver notifications, avoiding interruptions during active driving while ensuring timely delivery of safety-critical information. For example, users may receive voice based alerts to avoid any interaction with the phone. Moreover, the display of blood sugar will be on the car dashboard and hence, users may not have to hold their phone to look at blood sugar.

The feedback collection interface 154 may gather user responses to communications to refine future messaging strategies and improve user engagement. For example, if the system 100 detects that a user has a pattern of ignoring low blood sugars with a friendly tone (e.g., quiet, less intrusive audio), the feedback collection interface 154 may have the message tone change to try to improve engagement with the messages.

The monitoring system 100 may also include a dataset generator 156 for creating structured datasets from the aggregated anonymous user data. The dataset generator 156 may function to transform raw glucose and driving data into normalized, anonymized datasets suitable for research, analysis, and pattern detection across the user population. A “pattern” as used herein refers to a recognizable configuration of data points or events that, when detected, may indicate a condition relevant to driving safety or glucose management. In some embodiments, a pattern may be described by one or more of the following aspects:

Pattern {

| | |
|---|---|
| patternType: Enum | // Type identifier |
| temporalCharacteristics: Object | // Time-based attributes |
| glucoseCharacteristics: Object | // Glucose-based attributes |
| contextCharacteristics: Object | // Environmental/activity context |
| confidence: Number | // Detection confidence (0-1) |
| clinicalSignificance: Enum | // LOW, MEDIUM, HIGH, |
| | CRITICAL |

}

Example pattern may include types include, but are not limited to, temporal patterns, threshold violation patterns, rate-of-change patterns, and driving correlated patterns. An example temporal pattern may include post-meal glucose rise in which glucose increases following food consumption, typically occurring within approximately 30 to 90 minutes after eating. Another example temporal pattern may include dawn phenomenon in which glucose rises in early morning hours (approximately 4:00 am to 8:00 am) due to hormonal changes. Another example temporal pattern may include exercise-induced variation in which glucose changes are associated with physical activity, which may include initial decrease followed by delayed rise.

Threshold violation patterns may include a hypoglycemia episode in which glucose below approximately 70 mg/dL for a duration exceeding approximately 15 minutes; severe hypoglycemia in which glucose is below approximately 54 mg/dL; or a hyperglycemia episode in which glucose above approximately 180 mg/dL for an extended duration.

Rate-of-change patterns may include detecting: a rapid decline indicated by a rate of change exceeding approximately-2 mg/dL per minute for at least approximately 15 minutes; a rapid rise indicated by a rate of change exceeding approximately +2 mg/dL per minute for at least approximately 15 minutes; a glucose instability indicated by a high glucose variability indicated by coefficient of variation exceeding approximately 36 percent.

Driving-correlated patterns may include a pre-drive hypoglycemia tendency in which recurring pattern of glucose decline in temporal proximity to habitual driving times; a driving-associated glucose drop in which glucose decline that correlates with driving activity onset; or a route-specific patterns in which glucose behaviors associated with specific geographic routes or destinations.

The pattern detection techniques described herein may employ rule-based algorithms, machine learning classifiers, or hybrid approaches. In some embodiments, pattern detection may include performing a sliding window analysis in which continuous evaluation of glucose data within temporal windows of varying duration, performing a template matching in which comparison of current data to known pattern templates and/or performing anomaly detection in which identification of deviations from expected glucose behavior.

The dataset generator 156 may implement various data processing techniques, including temporal alignment of glucose readings with driving events, statistical normalization methods, and privacy-preserving anonymization protocols that maintain analytical value while protecting user identities. These datasets may be used internally to improve system algorithms, shared with the research community to advance understanding of diabetes management during driving activities, or provided to users for personal analysis and sharing with healthcare providers.

The monitoring system 100 may further include a ranking engine 158 that evaluates and prioritizes various elements within the system. The ranking engine 158 may score and rank insights based on relevance, novelty, and potential impact on user safety or glucose management. For example, a pattern showing consistent hypoglycemia after driving for more than 60 minutes may receive a higher relevance score than minor glucose fluctuations during short trips. The ranking engine 158 may also prioritize content recommendations, risk assessments, and community contributions based on factors such as statistical significance, user feedback, and clinical importance. This ranking functionality enables the system to present the most valuable information prominently, enhancing user experience and focusing attention on actionable insights with the greatest potential benefit. For example the ranking engine would determine that any glucose notification would be ranked higher than an app engagement or resource notification. This would be important for users when facing a hypoglycemic event so that they only received a notification about the clinically important event and would not be overwhelmed with other notifications.

The monitoring system 100 may further include a user interface (UI) generator 160. The UI generator 160 may generate one or more UI screens, as described in further detail elsewhere herein. The UI generator 160 may create dynamic, personalized interfaces that present glucose and driving data in intuitive visualizations tailored to individual user preferences and needs. These interfaces may include real-time glucose monitoring displays with driving status indicators, historical trend views comparing glucose patterns during driving versus non-driving periods, insight summaries highlighting key correlations, and interactive educational content. The UI generator 160 may employ responsive design principles to optimize display across various devices, including smartphones, tablets, and wearables, ensuring critical information remains accessible regardless of the user's preferred platform.

The ML models 162 (e.g., neural networks, ML models, training data 163, etc.) may be used to generate/determine personalized glucose behavior predictions and risk assessments for a user. For example, the systems and methods described herein may include the use of algorithms and/or ML models 162 to enable a user to review alerts and/or UI data including predicted glucose trends during planned driving activities, personalized risk scores for different driving scenarios, and recommended preventative actions. The ML models 162 may continuously learn from aggregated anonymous user data and individual user patterns to improve prediction accuracy over time. These models 162 may incorporate various data types, including historical glucose readings, driving durations and times, meal information, insulin dosing, and environmental factors to create comprehensive predictive frameworks. These frameworks implement a multi-layer architecture that first establishes user-specific baseline glucose dynamics through recurrent neural networks trained on temporal glucose patterns, then applies ensemble methods combining gradient-boosted decision trees and random forests to predict glucose trajectories during anticipated driving events. The framework incorporates a risk stratification layer that weighs multiple factors including glucose starting point, trajectory, driving duration, time of day, historical response patterns, and traffic conditions to generate a composite risk score on a 0-100 scale. This score is then mapped to specific intervention thresholds and recommendation categories through a rule-based expert system validated against clinically established hypoglycemia risk protocols. The system employs transfer learning techniques to apply population-level insights to new users while progressively adapting predictions to individual patterns as more user-specific data becomes available. The training data 163 may include validated datasets from controlled studies as well as anonymized real-world data from the user community, providing both scientific rigor and practical relevance to the model outputs.

In general, the ML models 162 may include neural networks that may be trained to perform constraint optimization modeling, regression modeling, similarity modeling, queue modeling, or other modeling technique to assess user patterns and/or trends and generate suggestions that fit the performed modeling. In some embodiments, the ML models 162 may utilize reinforcement learning models to learn user trends over time, for example. In some embodiments, the glucose prediction module employs one or more machine learning architectures 162 to forecast blood glucose trajectories over prediction horizons ranging from approximately 15 minutes to approximately 90 minutes. In some embodiments, the glucose prediction module employs one or more machine learning architectures 162 to forecast blood glucose trajectories over prediction horizons ranging from approximately 15 minutes to approximately 90 minutes. In some embodiments, the predictive model includes a hybrid convolutional-recurrent neural network architecture. The architecture may include a feature extraction stage with two groups of one-dimensional convolutional layers. Each group may include one or more convolutional layers followed by batch normalization. The convolutional layers may employ filter counts ranging from approximately 32 to approximately 128 filters, with kernel sizes ranging from approximately 3 to approximately 7. The architecture may further include a temporal modeling stage that has one or more Long Short-Term Memory (LSTM) layers. In some embodiments, the temporal modeling stage includes two LSTM layers, each having between approximately 64 and approximately 256 hidden units. In some embodiments, a dropout layer with a dropout probability between approximately 0.2 and approximately 0.5 may be positioned between LSTM layers to reduce overfitting.

In some embodiments, the glucose prediction algorithm may include generating a feature vector, extracting features, modeling the features, generating glucose predictions, and calculating a confidence level for the prediction and/or prediction uncertainty. For example, a first step may include feature vector construction for each prediction at time t. A construct input vector may be represented as X(t) as: X(t)=[G(t−n), G(t−n+1), . . . , G(t), EîG(t), EîG(t−1), . . . , D(t), T(t)] where G(t−i) represents glucose readings at time offsets; EîG(t−i) represents first-order glucose derivatives; D(t) represents binary driving state (e.g., 0=not driving, 1=driving); T(t) represents time-of-day encoding (e.g., sin/cos transformation); and n represents historical window length (e.g., 54 readings for 270-minute history). A second step may include convolutional feature extraction that applies one dimensional convolution operations represented by C_k(t)=œÊ(W_k*X(t)+b_k) where W_k represents learned kernel weights (e.g., size 3 \sqrt{ó1} to 7\sqrt{ó1}) which represents a convolution operator; and œE represents ReLU activation function k indexes filter number (e.g., 32 to 128 filters per layer). A third step may include an LSTM temporal modeling process to generate convolutional features through LSTM layers: h(t), c(t)=LSTM(C(t), h(t−1), c(t−1)) where h(t) represents hidden state vector (64-256 dimensions); c(t) represents cell state vector and LSTM implements standard gates: forget, input, output. A fourthe step may include generating predictions for glucose at an example horizon H (15-90 minutes): fú(t+H)=W_out¬Σh(t)+b_out where W_out represents output layer weights and fú (t+H) represents predicted glucose value at time t+H. A fifth step may include computing a confidence interval for a prediction uncertainly using ensemble variance: œÊ¬≤(t+H)=(1/M) OE£(fû_m (t+H)−·Π†(t+H))≤where M represents number of ensemble models (e.g., 5-10); fú_m represents prediction from model m; and Π† represents mean prediction across ensemble. This algorithmic implementation may provide a specific, repeatable technical process that transforms continuous glucose monitoring data into actionable driving safety predictions.

In some embodiments, the system 100 implements several hardware-specific optimizations to enable real-time glucose prediction on resource-constrained mobile devices. For memory management, the system 100 employs a sliding window buffer architecture that maintains the most recent N glucose readings in RAM, where N equals the prediction horizon in minutes multiplied by 3 and divided by the sampling interval in minutes, which may result in 54 readings for a 90-minute horizon with 5-minute sampling. Older readings may be compressed and stored in memory using delta encoding, where each stored value represents the difference from the previous reading, reducing storage requirements by approximately 60-70%.

To achieve computational efficiency with inference latency below 100 milliseconds on mobile processors, the system 100 implements quantization of neural network weights from 32-bit floating point to 8-bit integers, reducing model size by 75% while maintaining prediction accuracy within 2%. The system 100 may further combine batch normalization operations directly into convolutional layer computations through layer fusion and implements early exit mechanisms that halt computation when prediction confidence exceeds 95%.

For power optimization, the system 100 dynamically adjusts prediction frequency based on glucose stability and driving state. During stable non-driving periods, predictions occur every 15 minutes, while during driving with stable glucose, predictions occur every 5 minutes. When driving with unstable glucose, the system increases prediction frequency to every 1 minute. This adaptive scheduling reduces average power consumption by approximately 40% compared to fixed-interval prediction approaches.

The real-time prediction of glucose trajectories during driving utilizes computational capabilities that cannot be performed by human mental processes. The prediction algorithm processes 54 historical glucose readings through 32-128 convolutional filters, applies LSTM temporal modeling with 64-256 hidden units, and generates ensemble predictions with confidence intervals and all within 100 milliseconds. This computational throughput, combined with the mathematical complexity of multi-layer neural network operations involving thousands of weighted connections and non-linear activation functions, is achievable only through specialized computer processors. Moreover, the continuous learning and model updating based on federated learning across thousands of users represents a computational task that fundamentally requires networked computer systems and cannot be replicated through manual analysis or traditional medical monitoring approaches.

The system 100 interfaces with vehicle telematics through a hardware abstraction layer that supports multiple protocols for sensor fusion hardware integration. This includes a CAN bus interface for direct OBD-II communication complying with ISO 15765-4 standards, Bluetooth Low Energy (BLE 5.0) for wireless vehicle connectivity, and USB-C with support for both USB 2.0 and USB 3.1 for wired connections. These hardware-specific implementations provide concrete technical solutions to the computational challenges of real-time glucose prediction during driving.

The architecture may further include an output stage that has one or more fully connected layers. The output layer configuration may vary based on prediction horizon, with example configurations including: approximately 32 neurons for a 30-minute horizon, approximately 32 to 64 neurons for a 60-minute horizon, and approximately 64 neurons for a 90-minute horizon.

In some embodiments, the predictive model utilizes a Temporal Fusion Transformer architecture, which may provide advantages for deployment on resource-constrained mobile devices due to reduced computational requirements during inference. In some embodiments, the predictive model utilizes an ensemble of multiple model architectures, in which predictions from individual models are combined using weighted averaging. Weights may be determined using optimization techniques such as genetic algorithms, with individual model weights ranging from approximately 0.05 to approximately 0.5.

The predictive model may be trained using historical continuous glucose monitoring (CGM) data. In some embodiments, training employs an RMSProp optimizer with a learning rate between approximately 0.00001 and approximately 0.001. Training may proceed for up to approximately 500 epochs with early stopping based on validation loss.

The input to the predictive model may include a temporal window of glucose readings. In some embodiments, the input window length is approximately three times the prediction horizon. For example, a 90-minute prediction horizon may utilize an input window of approximately 270 minutes of historical glucose data.

In some embodiments, the system 100 may employ transfer learning to personalize predictions for individual users. A base model is pre-trained on population-level data, and personalization is achieved by fine-tuning model parameters using individual user data. In some embodiments, effective personalization may be achieved with as few as approximately 5 to approximately 10 days of individual user data. This timeline advantageously aligns with typical CGM sensor replacement cycles, enabling personalization to occur during initial system use.

In some embodiments, the system 100 may employ federated learning techniques in which model updates are computed locally on user devices and aggregated without transmitting raw glucose data. This approach may employ the Federated Averaging algorithm, where a fraction C of available clients (where C may be approximately 0.1) are selected for each training round, each selected client computes local model updates, the updates are aggregated at a central server using weighted averaging based on local dataset sizes, and the aggregated model is distributed back to clients

In some embodiments, training employs a loss function that applies differential weighting to prediction errors in clinically significant glucose ranges. Errors in hypoglycemic ranges (below approximately 70 mg/dL) and hyperglycemic ranges (above approximately 180 mg/dL) may receive higher penalty weights than errors in euglycemic ranges.

In some embodiments, the ML models 162 may be trained using training data 163. The training data 163 may include, but is not limited to logs of historical user data and/or other user data, continuous glucose monitoring (CGM) readings, driving event timestamps, trip durations, time-of-day information, meal logging data, insulin administration records, user demographic information, vehicle telematics data, and/or user feedback on system-generated insights, etc. In some embodiments, training data 163 may be collected by secure API integrations with glucose monitoring devices, mobile device motion sensors, vehicle connectivity systems, and user self-reporting through the application interface. Such data may be anonymized to remove identifiable information before being used as training data. In one non-limiting example, the ML models 162 may be trained on historical glucose patterns and driving session data for any number of users to predict potential hypoglycemic or hyperglycemic events during future driving sessions for a user.

The ML models 162 may generate algorithms that use machine learning techniques to generate insights, patterns, and trends for a user. These techniques may include supervised learning methods for classification of driving-related glucose events, unsupervised learning for pattern clustering across user populations, reinforcement learning for optimizing notification timing, and deep learning approaches for complex multivariate analysis of physiological responses to driving conditions. The algorithms may identify correlations between glucose stability and factors such as trip duration, time of day, traffic conditions, pre-driving glucose levels, and recent insulin or carbohydrate intake. The ML models 162 may also apply time-series analysis to detect recurring patterns in glucose variability during specific driving scenarios and generate personalized risk profiles that evolve as the system continues to learn from individual user data. These insights may be continuously refined through feedback loops that incorporate user confirmation of detected patterns and the effectiveness of recommended interventions.

In some embodiments, the ML models 162 illustrated in FIG. 1 may employ transfer learning to personalize predictions for individual users. A base model is pre-trained on population-level data, and personalization is achieved by fine-tuning model parameters using individual user data. In some embodiments, effective personalization may be achieved with as few as approximately 5 to approximately 10 days of individual user data. This timeline advantageously aligns with typical CGM sensor replacement cycles, enabling personalization to occur during initial system use. In some embodiments, the system employs federated learning techniques wherein model updates are computed locally on user devices and aggregated without transmitting raw glucose data. This approach may employ the Federated Averaging algorithm, where a fraction C of available clients (where C may be approximately 0.1) are selected for each training round, each selected client computes local model updates, the updates are aggregated at a central server using weighted averaging based on local dataset sizes, and the aggregated model is distributed back to clients.

In some embodiments, the ML models 162 illustrated in FIG. 1 may be configured to perform anomaly detection to identify unusual glucose responses during driving that deviate from a user's established baseline patterns. The system may implement recurrent neural networks (RNNs) to analyze temporal sequences of glucose readings during drives, capturing the dynamic nature of glucose changes over time. Gradient boosting algorithms may be employed to identify the relative importance of different factors contributing to glucose variability while driving. The system may utilize k-means clustering to group similar driving experiences based on glucose outcomes, helping users recognize high-risk scenarios. Dynamic time warping algorithms may compare glucose patterns across different driving events of varying durations to identify consistent response patterns regardless of trip length. Seasonal trend decomposition methods may separate recurring patterns from random variations in glucose response to driving, highlighting meaningful trends. The system may also implement Bayesian networks to model probabilistic relationships between driving conditions, user actions, and glucose outcomes, enabling more robust predictive capabilities in complex scenarios with uncertain information.

The monitoring system 100 may include one or more processors 164 and memory 166. The processors 164 may include one or more processors that include one or more devices capable of executing instructions, such as instructions stored by the memory 166, to perform operations and/or communications amongst systems, engines, modules, and/or devices described herein.

The memory 166 can include one or more non-transitory computer-readable storage media. The memory 166 may store instructions and data that are usable in combination with processors 164 to execute the processes and/or algorithms described herein as well as to execute or interface with ML models 162, insight generation engine 124, risk assessment engine 134, communication engine 144, UI generator 160, dataset generator 156, and/or ranking engine 158. The memory 166 may also function to store or have access to the ML models 162, insight generation engine 124, risk assessment engine 134, communication engine 144, UI generator 160, dataset generator 156, and/or ranking engine 158

Additional apps 168 may also be integrated into monitoring system 100. For example, targeted advertisement applications may deliver relevant diabetes management products based on user insights; insurance applications may provide premium discounts for demonstrating responsible glucose management while driving; ride-sharing services may integrate to suggest alternative transportation when glucose levels indicate elevated risk; existing CGM apps and insulin pump applications may exchange data bidirectionally to create a unified diabetes management ecosystem; restaurant applications may suggest appropriate meal options based on current glucose trends and planned driving activities; and navigation services may incorporate glucose-aware route planning that includes recommended rest stops based on predicted glucose patterns. These third-party integrations extend the system 100 core capabilities through a secure API framework that maintains user privacy while enabling a comprehensive diabetes management experience that seamlessly addresses the unique challenges of driving with diabetes. Each integration undergoes rigorous validation by way of a data validation module 138 to ensure data security and functional reliability before being made available to users, with clear consent mechanisms for controlling data sharing permissions across the application ecosystem.

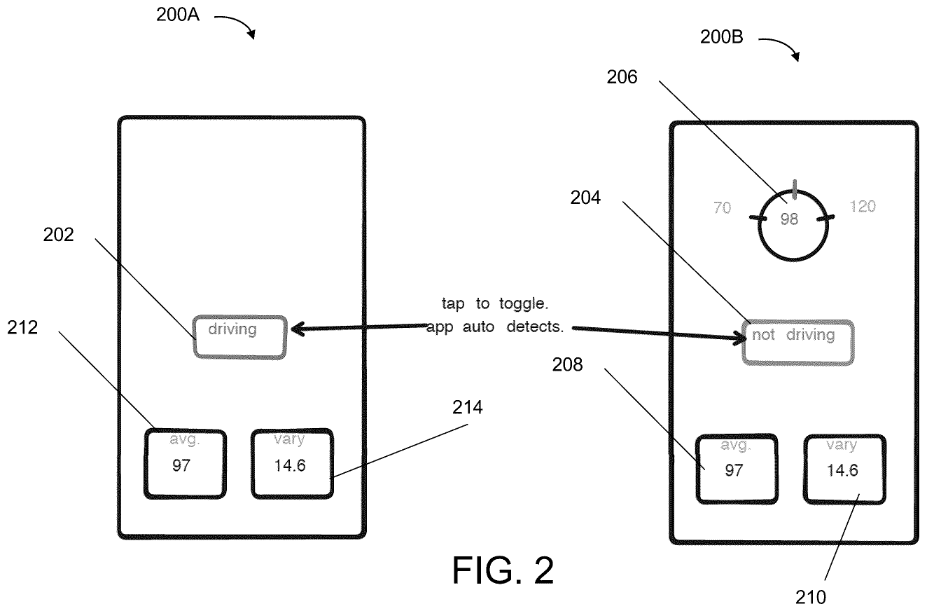

FIG. 2 illustrates example user interfaces 200A and 200B of the monitoring system 100 displaying blood glucose insights related to a driving status. The interfaces 200A and 200B feature visual indicators of a current driving state of the user. In this example, interface 200A depicts that the user is driving, at control 202. Similarly, interface 200B indicates that the user is not driving at the control 204. Controls 202, 204 may be toggled by the user. In some embodiments, the controls 202, 204 may instead be modified in response to the system 100 (e.g., event detector 148) automatically detecting the driving state of a vehicle being operated by the user and modifying the display in controls 202, 204 based on the detected driving state. The user interface 200B depicts a glucose monitoring value 206 (e.g., 98 mg/dL) prominently displayed in a circular gauge with threshold indicators at 70 mg/dl and 120 mg/dL to provide immediate visual context of safety ranges for the glucose level of the user. The user interface 200B also displays average glucose values 208 (e.g., 97 mg/dL) and glucose variability measurements 210 (e.g., 14.6) for convenience to the user. The user interface 200A depicts an average glucose value 212 (e.g., 97 mg/dL) and the variability measurements 214 (e.g., 14.6), but does not depict the current glucose monitoring value 206 to avoid distracting the user while driving.

The driving state detection functionality can be implemented through multiple approaches. For example, the monitoring system 100 may utilize a GPS of a mobile device (paired with the vehicle or a mobile device unpaired with the vehicle or a GPS of the vehicle) and accelerometer data of the mobile device to detect vehicle motion patterns consistent with driving. This can be accomplished by analyzing speed, trajectory patterns, and motion signatures characteristic of vehicular travel. Alternatively, the system 100 may integrate with vehicle telematics systems using Bluetooth or direct connections to onboard diagnostic (OBD) ports to definitively determine driving status. The system 100 may also provide manual toggle capabilities to accommodate situations in which automatic detection may not function optimally. When a driving state change is detected, the system may begin correlating continuous glucose monitoring (CGM) data (e.g., BG levels 104) from sensors 106 with the driving event, establishing precise temporal boundaries for subsequent analysis by the insight generation engine 124.

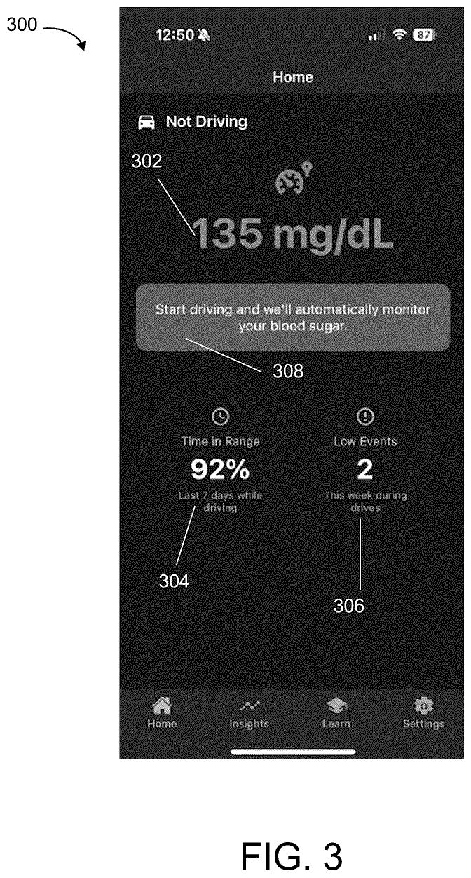

FIG. 3 depicts another example user interface 300 of the monitoring system 100 in a non-driving state, showing the current glucose reading 302 (135 mg/dL) along with performance metrics (not shown) related to glucose management during driving. The interface 300 may include a call-to-action button 308 encouraging the user to begin driving, which triggers the automatic monitoring sequence. Key performance indicators are displayed, including time in range 304 (e.g., 92 percent) while driving over the past 7 days and the number of low glucose events 306 (e.g., 2) occurring during drives within the current week.

The technical implementation of the interface 300 relies on persistent storage of historical driving and glucose data, which is processed through the pattern/trend determination engine 128 to generate these metrics. The system 100 may maintain a continuous connection to CGM data sources through secure APIs (such as Dexcom's API as mentioned elsewhere herein) to receive real-time glucose values with timestamps. The historical blood glucose data may be filtered and segmented based on previously recorded driving events, allowing the system 100 to calculate specific driving-related glucose metrics. The 92 percent time-in-range metric 304 is computed by determining the percentage of glucose readings that fell within the user's predefined safe glucose thresholds during all driving periods within the past seven days. Similarly, the low event count 306 is determined by analyzing glucose data streams during driving periods and identifying instances where values fell below the user's configured low threshold or exhibited rapid downward trends potentially indicative of hypoglycemic events.

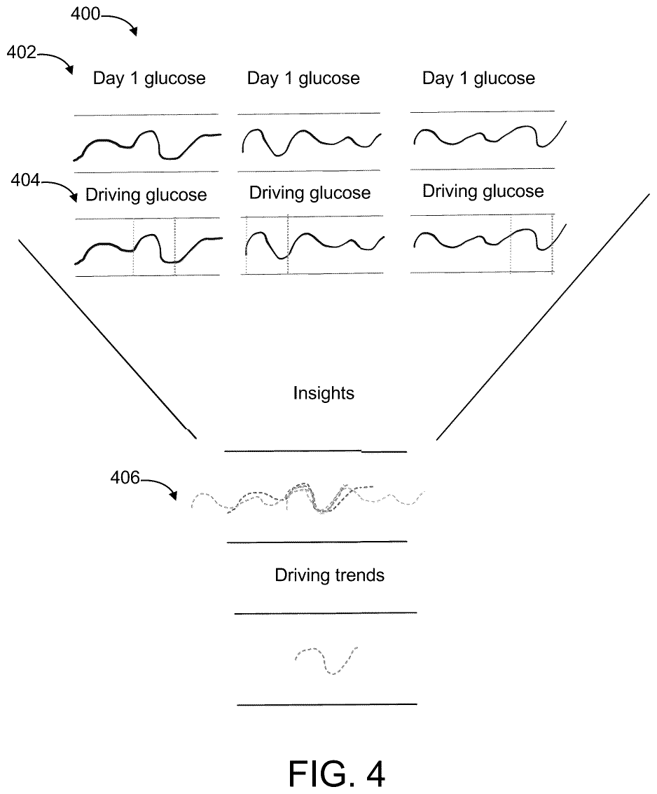

FIG. 4 illustrates example graphical insights and driving trends interface 400 generated by the monitoring system 100. The interface 400 includes a comparative visualization approach with paired data: general daily glucose patterns (top row 402) juxtaposed with driving-specific glucose patterns (middle row 404). The bottom section includes one or more visualization 406 that displays aggregated driving trends derived from pattern analysis across multiple driving events. Red dotted lines on the driving glucose charts indicate temporal boundaries of driving events, enabling clear visualization of glucose behavior specifically during vehicle operation.

This visualization 406 is generated through sophisticated data processing performed by the insight generation engine 124 and the pattern/trend determination engine 128. The system processes and aligns temporal glucose data from sensors 106 with driving event timestamps stored in the user data 102. The comparative analytics functionality implements algorithms that identify statistically significant deviations in glucose patterns during driving compared to non-driving periods. The analysis module 126 applies time-series analysis techniques to detect recurring patterns specific to driving durations, times of day, and routes. These patterns are then clustered and processed using machine learning techniques as described elsewhere herein, including gradient boosting algorithms to identify relative importance of factors contributing to glucose variability while driving, and k-means clustering to group similar driving experiences based on glucose outcomes. The visualization 406 is dynamically generated by the UI generator 160, which implements responsive design principles to ensure appropriate display across various device form factors while maintaining the critical comparative aspects needed for user comprehension.

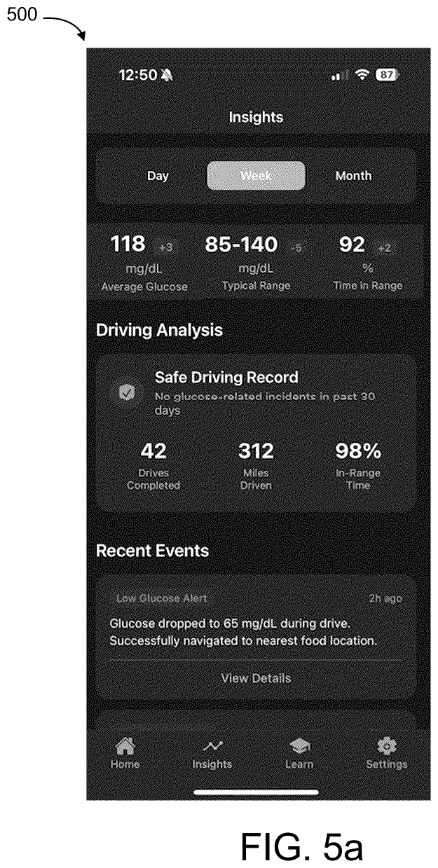

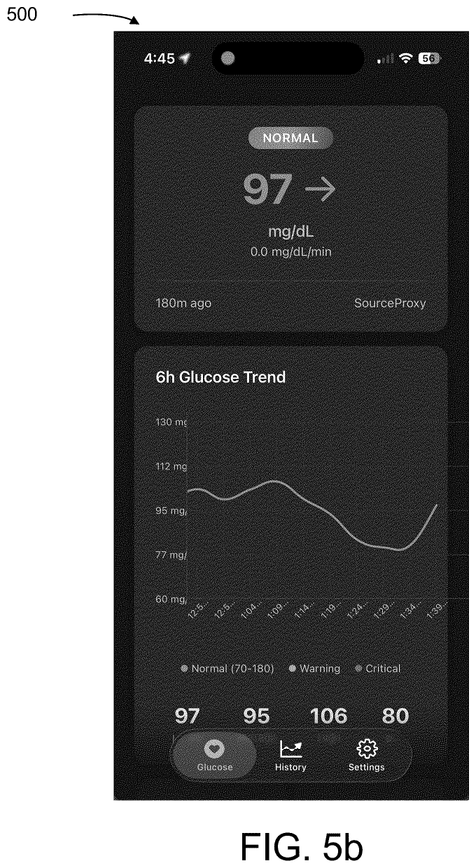

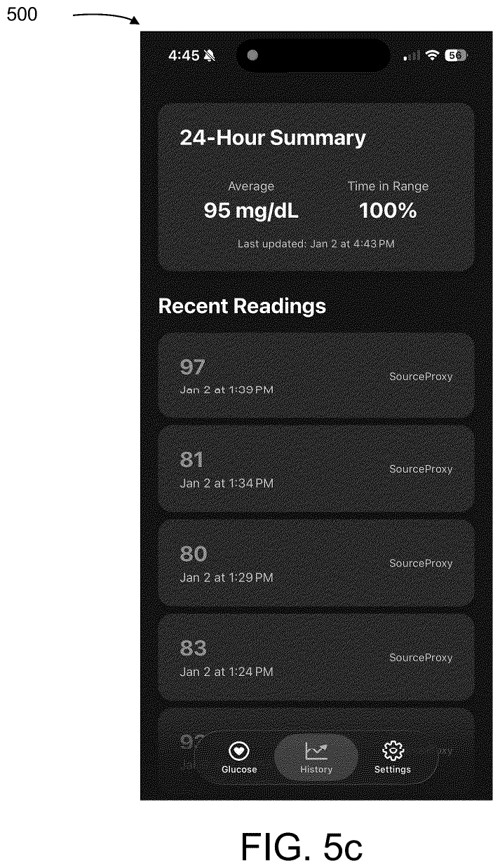

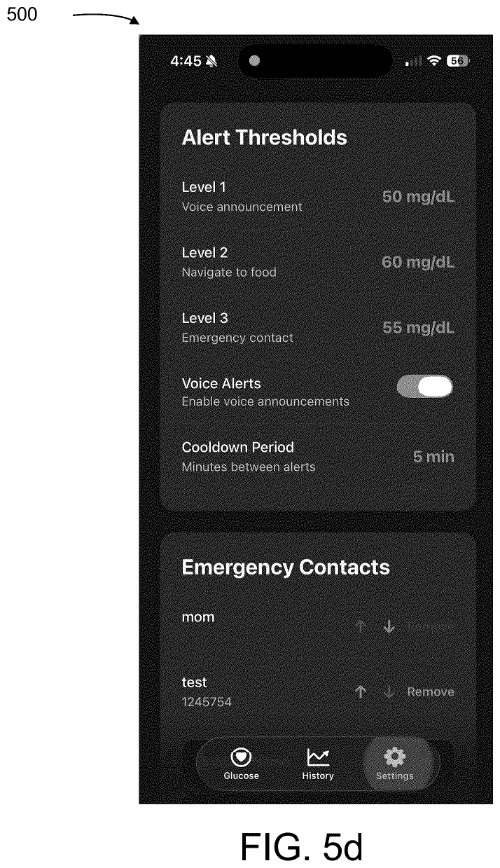

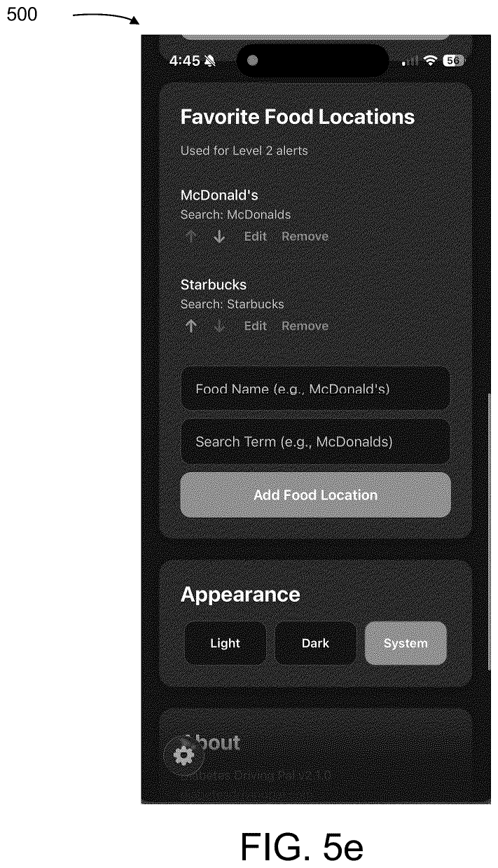

FIG. 5a illustrates an example insights interface 500 providing comprehensive analysis of the user's glucose management specifically during driving activities. The interface presents critical metrics including average glucose (118 mg/dL), typical glucose range (85-140 mg/dL), and time in range percentage (92 percent), all accompanied by trend indicators showing change from previous periods. The driving analysis section prominently features safety information, including a “Safe Driving Record” confirmation with no glucose-related incidents in the past 30 days, along with driving statistics such as completed drives (42), miles driven (312), and in-range time percentage (98 percent). The interface also displays a chronological list of recent events, such as a low glucose alert that occurred 2 hours ago, with details about the system's intervention and the outcome.

This interface is powered by the risk assessment engine 134 working in conjunction with the insight generation engine 124. The glucose statistics are calculated through statistical analysis performed on temporally filtered CGM data corresponding precisely to driving events. The driving analysis module 136 specifically analyzes factors unique to the driving environment that may impact glucose levels, such as traffic stress, time of day, duration, and frequency of stops. The safe driving record status is determined through the event detector 148, which applies predefined threshold criteria against the user's glucose and driving history to identify any incidents that would constitute a safety concern. The system uses the ranking engine 158 to evaluate and prioritize insights based on relevance, novelty, and potential impact on user safety, ensuring that the most valuable information is prominently displayed. The “Successfully navigated to nearest food location” indicator demonstrates the system's integration with navigation services and location-based APIs to guide users to appropriate resources when needed, a capability described elsewhere herein.

FIG. 5B illustrates an example home screen interface 510 of the monitoring system 100 displaying real-time glucose monitoring with driving mode integration. The interface prominently features a current glucose value display 512 rendered in large typography (e.g., 48-point font) for immediate visibility, accompanied by a trend indicator arrow 514 showing the directional movement of glucose levels (e.g., rising, falling, stable). The trend indicator may display various arrow symbols including upward arrows for rising glucose, downward arrows for falling glucose, and horizontal arrows for stable readings. A status badge 516 provides color-coded context using gradient backgrounds: green for normal glucose range (70-180 mg/dL), orange for warning levels, and red for critical levels requiring immediate attention.

The interface 510 includes a glucose trend chart 518 displaying a six-hour sliding window of historical glucose data. The chart renders data points with color coding corresponding to glucose status: green points for values within normal range, orange points for warning thresholds, and red points for critical values. The visualization includes a smooth curve connecting data points and displays summary statistics including the latest reading, average glucose, highest value, and lowest value within the displayed time period.

Quick statistics cards 520 are presented below the trend chart, displaying 24-hour average glucose values and time-in-range percentage calculated against a target range (e.g., 70-180 mg/dL). These metrics are generated through the analysis module 126 processing temporal glucose data from sensors 106.

When the system detects that the user is operating a vehicle, the interface 510 transitions to a driving mode configuration displaying a driving mode banner 522 in high-contrast warning colors. The banner displays the current vehicle speed (e.g., “DRIVING MODE-XX mph”) and session duration to provide contextual awareness. In driving mode, the interface simplifies by hiding non-critical statistics and enlarging the glucose value display for improved glanceability while driving. The driving state detection functionality utilizes GPS and accelerometer data to detect vehicle motion patterns consistent with driving, automatically activating when sustained speeds exceed a threshold (e.g., 9 mph). Manual toggle controls 524 allow users to start or stop driving mode when automatic detection is not optimal.

Information cards 526 displayed in non-driving mode educate users about the automatic driving detection capability and the three-level alert system, including Level 1 voice announcements at configurable thresholds (e.g., 70 mg/dL), Level 2 navigation assistance to nearby food locations (e.g., 60 mg/dL), and Level 3 emergency contact activation (e.g., 55 mg/dL).

FIG. 5C illustrates an example history interface 530 of the monitoring system 100 providing detailed glucose reading history and analytics. The interface presents a 24-hour summary card 532 displaying aggregate metrics including average glucose over the past 24 hours, time-in-range percentage indicating the proportion of time glucose values remained within a target range (e.g., 70-180 mg/dL), and a timestamp indicating when statistics were last calculated.

The interface 530 includes a recent readings list 534 displaying glucose measurements in reverse chronological order. Each reading entry 536 presents the glucose value with color coding corresponding to glucose status: red for critical values below a severe threshold (e.g., 55 mg/dL), orange for low values below a warning threshold (e.g., 70 mg/dL), green for values within normal range (e.g., 70-180 mg/dL), orange for high values exceeding an upper threshold (e.g., 180 mg/dL), and red for very high values exceeding a critical upper threshold (e.g., 250 mg/dL). Each entry further displays a trend arrow 538 indicating glucose direction, a formatted timestamp 540 (e.g., “Jan 15, 3:42 PM”), a trend rate 542 showing the rate of change in mg/dL per minute with positive or negative indicators, and a source identifier 544 indicating the device or application from which the reading originated.

The history interface implements pull-to-refresh functionality to retrieve the latest glucose data from connected health data sources (e.g., Apple HealthKit). The analysis module 126 processes historical glucose readings to calculate the displayed statistics, filtering and segmenting data based on time periods to generate meaningful metrics. This historical data serves as input to the pattern/trend determination engine 128 for identifying recurring patterns in glucose behavior, which may be correlated with driving events stored in user data 102 to generate personalized insights.

FIG. 5D illustrates an example settings interface 550 of the monitoring system 100 enabling user configuration of alert thresholds and emergency contacts. The interface presents an alert thresholds section 552 allowing users to customize the glucose levels at which different alert types are triggered. Three configurable threshold levels are displayed: a Level 1 threshold 554 for voice announcements (defaulting to 70 mg/dL), a Level 2 threshold 556 for navigation to food locations (defaulting to 60 mg/dL), and a Level 3 threshold 558 for emergency contact activation (defaulting to 55 mg/dL). Each threshold displays the current value and a descriptive label indicating the alert action. Users may tap on any threshold to access an edit interface with numeric input validation constraining values to a safe range (e.g., 40-100 mg/dL).

The interface 550 includes a voice alerts toggle 560 allowing users to enable or disable voice announcements, addressing user preferences for notification methods as described in user inputs 110. A cooldown period display 562 indicates the minimum time between repeated alerts (e.g., 5 minutes) to prevent alert fatigue, implementing the functionality of the notification prioritization module 150 which determines relative importance of messages to ensure critical safety notifications are distinguished from informational updates.

An emergency contacts section 564 displays a list of user-designated contacts to be notified when glucose reaches critical levels. Each contact entry 566 shows the contact name and phone number, with priority ordering controls 568 (up and down arrows) allowing users to arrange contacts in order of notification preference. The highest-priority contact is called first when a Level 3 alert is triggered. Remove buttons 570 enable deletion of contacts with confirmation dialogs to prevent accidental removal. An add contact form 572 includes text input fields for contact name and phone number, with validation ensuring complete information before addition. These emergency contacts are stored in user data 102 and accessed by the communication engine 144 when initiating emergency calling protocols as described elsewhere herein.

FIG. 5E illustrates an example settings interface 580 of the monitoring system 100 enabling user configuration of favorite food locations and application appearance. The interface presents a favorite food locations section 582 used for Level 2 alerts, implementing the technical solution described elsewhere herein for providing automated navigation to nearby food establishments based on stored user preferences for glucose-stabilizing options.

The favorite food locations list 584 displays user-configured food establishments with each entry showing a display name 586 (e.g., “McDonald's”, “Starbucks”, “Subway”) and a search term 588 used for navigation queries (e.g., “McDonalds”). Priority ordering controls 590 allow users to reorder food locations, with higher-priority locations presented first when the system initiates navigation assistance during a Level 2 alert. Edit buttons 592 enable modification of existing entries through an inline edit form with text inputs for name and search term. Remove buttons 594 enable deletion with confirmation. An add food location form 596 includes input fields for food name and search term, allowing users to expand their personalized list of glucose-stabilizing food options. When a Level 2 alert is triggered, the safety recommendation generator 142 utilizes this stored preference data in conjunction with mapping service APIs to identify and display the nearest matching food establishment along the user's current route.

The interface 580 further includes an appearance section 598 presenting theme selection options. A three-button toggle interface 600 enables selection between light mode (featuring a cream-colored background with dark text), dark mode (featuring a dark background with light text), and system mode (automatically following the device's system-wide appearance setting). The active theme selection is visually distinguished through color highlighting. These appearance preferences are stored locally and applied through the UI generator 160, which employs responsive design principles to optimize display across various devices while maintaining accessibility and readability in different lighting conditions, including nighttime driving scenarios.

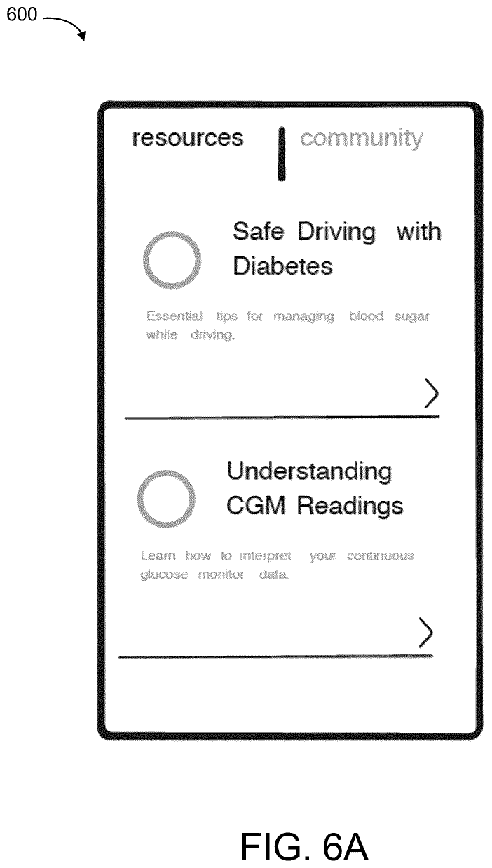

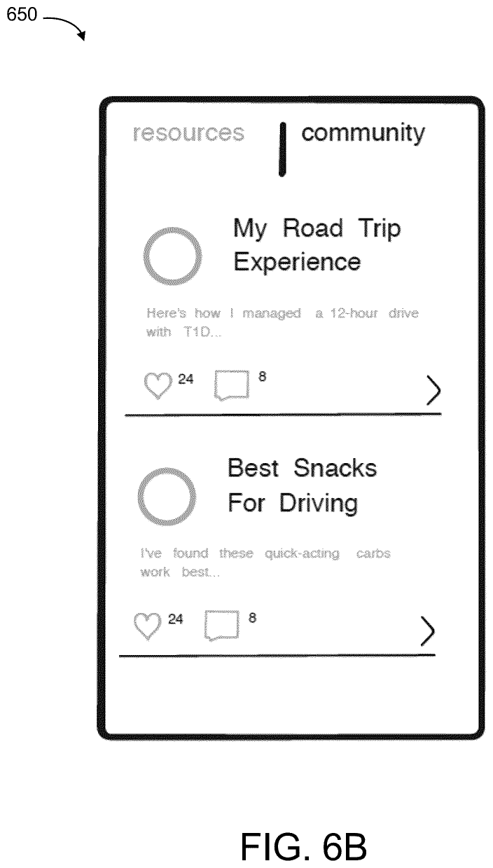

FIG. 6A illustrates an example user interface 600 depicting featured resources corresponding to insights generated for the user. The interface presents educational content in a tabbed view, with “resources” actively selected and “community” as an alternative tab. Two featured resource cards are displayed: “Safe Driving with Diabetes” and “Understanding CGM Readings,” each providing information relevant to the user's glucose management while driving.

The technical implementation of this resource delivery system involves several components of the monitoring system 100. The content/link generator 130, may generate personalized educational content based on identified patterns to help users better manage their condition while driving. The system determines which resources to display by analyzing the user's specific glucose patterns and driving behaviors through the insight generation engine 124 and risk assessment engine 134. The ranking engine 158 scores and prioritizes content based on relevance to the user's specific situation, statistical significance, and clinical importance. For example, if pattern analysis reveals consistent post-driving hyperglycemia, resources addressing this specific pattern would receive higher priority. The resources are delivered through a secure API framework that maintains user privacy while enabling comprehensive diabetes management. The educational content is dynamically generated or selected from a curated database of medically reviewed information, ensuring accuracy and relevance to the specific insights identified for the user.

FIG. 6B illustrates an example user interface 650 depicting featured community-generated content, displayed when the user selects the “community” tab. The interface showcases user-contributed experiences and advice related to managing diabetes while driving, including “My Road Trip Experience” from a user with Type 1 diabetes describing a 12-hour drive, and “Best Snacks For Driving” featuring quick-acting carbohydrate recommendations. Each community post includes engagement metrics such as likes (24) and comments (8), and provides navigation to full content through chevron icons.

This community content functionality is enabled by a secure crowd-sourced data platform described elsewhere herein, which allows users to securely and voluntarily share insights and experiences. The technical implementation involves anonymized data aggregation through the dataset generator 156, which transforms raw glucose and driving data into normalized, anonymized datasets suitable for pattern detection across the user population, as described elsewhere herein. Community content undergoes validation through the data validation module 138 described elsewhere herein, which verifies the accuracy and reliability of user-contributed information before it becomes available to the broader community. The ranking engine 158 evaluates community contributions based on factors such as statistical significance, user feedback, and clinical importance to prioritize the most valuable content, as described elsewhere herein. This community-sourced knowledge base serves as a supplementary resource to the system-generated insights, providing real-world experiences and practical solutions that complement the data-driven analytics provided by the monitoring system 100.

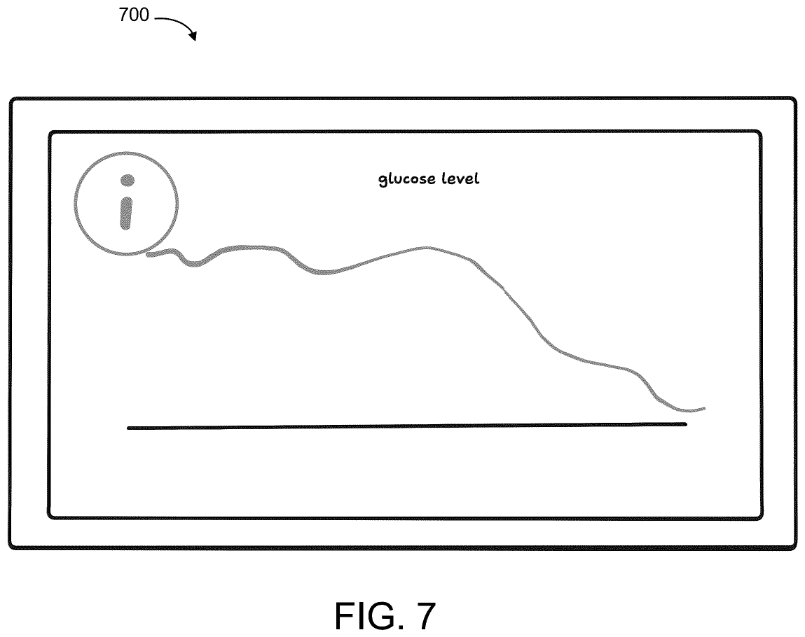

FIG. 7 illustrates an example first level alert 700 representing a mildly lower blood sugar level while driving. The interface displays an information icon and a glucose trend line that shows the current directional movement of the user's glucose levels. This first-level alert is designed to provide an early warning to the driver before glucose levels reach potentially dangerous thresholds, allowing for proactive intervention.

The technical implementation of this alert system involves continuous real-time processing of glucose data from CGM systems through secure API connections. The predictive alert module 140 forecasts potential glucose events based on historical patterns and current trends, enabling preemptive notifications before the user reaches unsafe glucose levels while driving. The alert/message generator 146 produces contextually appropriate messages regarding glucose levels and driving safety, using language and urgency levels tailored to the specific situation, as described elsewhere herein. When the system detects glucose levels trending toward the lower boundary of the safe range (defaulting to 90 mg/dL if the user has not selected an individualized target, it triggers this first-level alert. The alert is delivered through voice-enabled text to minimize driver distraction, utilizing the delivery timing optimizer 152 to select appropriate moments that avoid interruptions during active driving while ensuring timely delivery of potentially important information. The system can integrate with vehicle audio systems through Bluetooth connections or car interface platforms like Apple CarPlay and Android Auto to deliver these voice alerts through the vehicle's speakers.

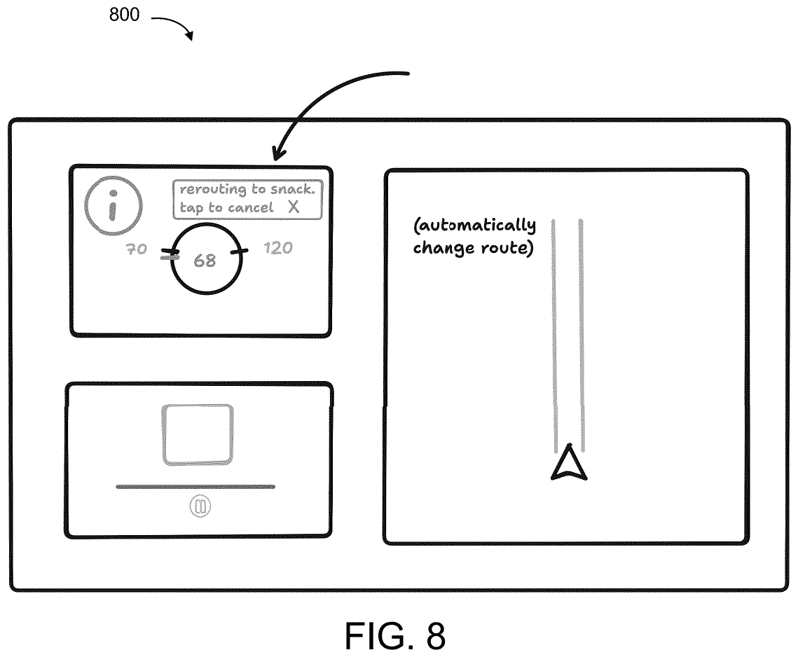

FIG. 8 illustrates an example second level alert 800 triggered when blood glucose falls below 70 mg/dL while driving. The interface displays a more urgent warning with the text “rerouting to snack” and the current glucose value (68 mg/dL) prominently displayed in red, indicating a level requiring immediate attention. The right portion of the interface shows a navigation map that automatically initiates rerouting to nearby food locations where the driver can safely stop and treat the low blood glucose with appropriate food.

This second-level alert functionality is implemented through a coordinated system of components described in the patent. The driving analysis module 136 continuously monitors glucose levels in relation to the driving environment, analyzing factors such as traffic conditions and available stopping points. When blood glucose drops to 70 mg/dL or below (the predefined/default level as described elsewhere herein), the safety recommendation generator 142 produces customized safety guidelines for the individual user based on their specific glucose response patterns to driving. The navigation functionality represents the technical solutions described herein, which provides automated navigation to nearby food establishments based on stored user preferences for glucose-stabilizing snacks. This is accomplished through integration with mapping services such as Google Maps or Apple Maps via their respective APIs, with additional data processing to prioritize locations based on proximity to the current route, availability of appropriate food options, and ease of access from the roadway. The system can transmit this navigational data to the vehicle's infotainment system through Apple CarPlay or Android Auto integration, providing turn-by-turn directions with minimal driver interaction required. This implementation improves safety by encouraging drivers to pull over and treat low blood sugar before it reaches a critically low level which can impair judgment and brain functions, as described elsewhere herein.

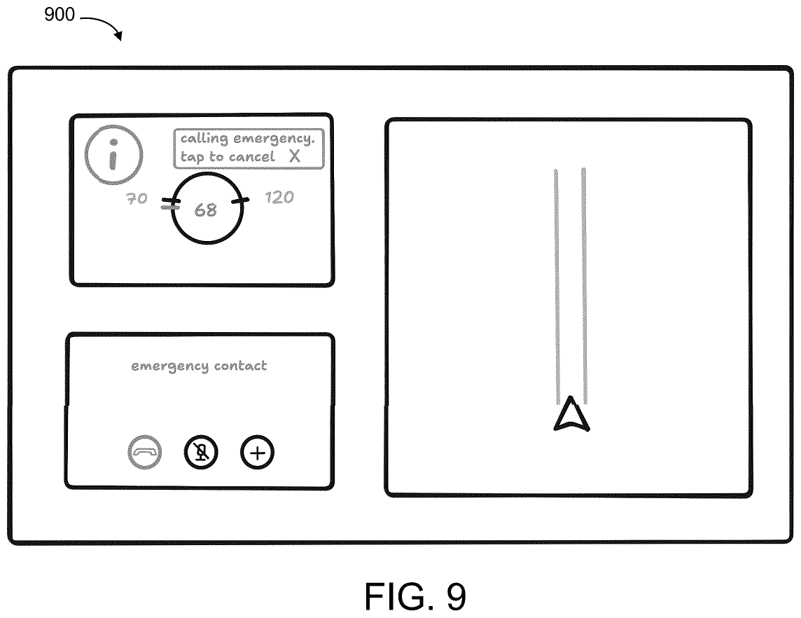

FIG. 9 illustrates an example third level alert 900 triggered when blood glucose reaches a critical low level while driving. The interface prominently displays “calling emergency” with a cancellation option, and shows the current glucose reading (68 mg/dL) in red. The lower panel displays emergency contact options with vehicle-appropriate controls designed for easy access during a potential crisis situation. This represents the most severe alert level in the system, activated when blood glucose approaches levels that could significantly impair driving ability.

The technical implementation of this critical alert system addresses the emergency response coordination problem described elsewhere herein. When glucose levels approach or fall below 54-55 mg/dL, considered critical low blood sugar, the system initiates its emergency protocols. The event detector 148 identifies this as a significant event warranting urgent communication, and the alert prioritization module 150 assigns the communication the highest priority to ensure it overrides any less critical notifications. The system automatically initiates emergency calling protocols to ensure driver safety, contacting either pre-selected emergency contacts (friends or family members) or emergency services such as 911, as described elsewhere herein. This functionality is implemented through direct integration with the mobile device's telephony system or the vehicle's emergency call system if available. The automated emergency response system monitors both the severity and duration of glucose excursions during driving and establishes communication protocols with emergency services, automatically transmitting relevant medical information and current location data when predetermined risk thresholds are exceeded, as described elsewhere herein. Location data is obtained through GPS and transmitted along with the user's medical information to emergency services, enabling faster and more effective response. This integration can work through vehicle GPS or satellite systems or through smartphone emergency protocols, depending on the available technology.

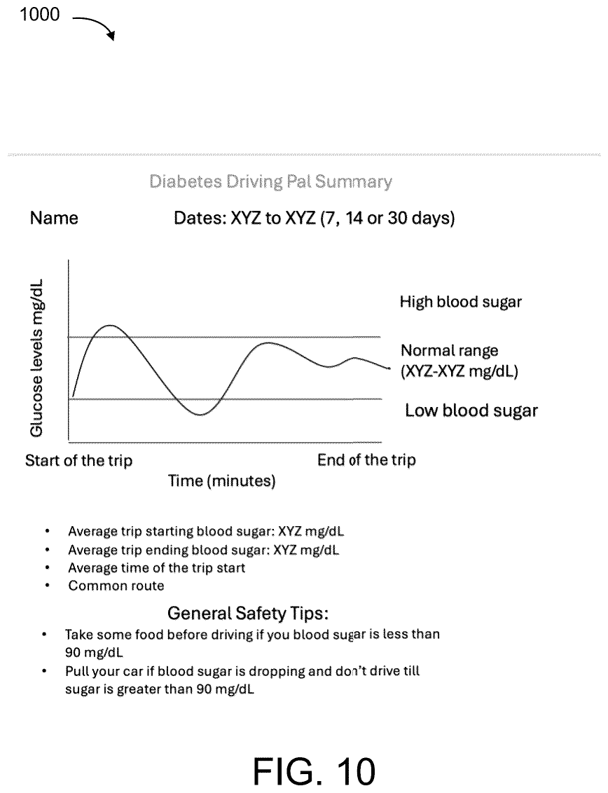

FIG. 10 illustrates example download/export features 1000 that users can share with their healthcare providers or other users. The figure shows a comprehensive summary report titled “Diabetes Driving Pal Summary” that includes the user's name, date range selection options (7, 14, or 30 days), and a visual representation of glucose levels throughout a typical driving period. The report displays high, normal, and low blood sugar ranges, with a glucose trend line showing fluctuations from trip start to end. Key metrics are provided, including average trip starting and ending blood glucose values, average trip start time, and common routes, along with general safety tips for diabetes management while driving.

This report generation functionality is implemented through the dataset generator 156, which transforms raw glucose and driving data into normalized, structured datasets suitable for analysis and sharing. The system enables users to create custom datasets through an intuitive dataset builder with preset filters and parameters to help users focus on relevant data pertaining to diabetes management before, during, and after driving activities. The technical implementation involves several data processing techniques described herein, including temporal alignment of glucose readings with driving events, statistical normalization methods, and privacy-preserving anonymization protocols that maintain analytical value while protecting user identities. The system applies time-series analysis to the collected data, detecting recurring patterns in glucose variability during specific driving scenarios. Export capabilities support common formats compatible with popular analysis tools, enabling additional research or sharing of findings with healthcare providers. The visualization components are generated through the UI generator 160, which creates dynamic, personalized interfaces tailored to individual user preferences and needs. This report can be exported in various formats (PDF, CSV, etc.) and shared electronically with healthcare providers through secure channels, or printed for in-person consultations, providing valuable insights into how driving affects the user's glucose management.

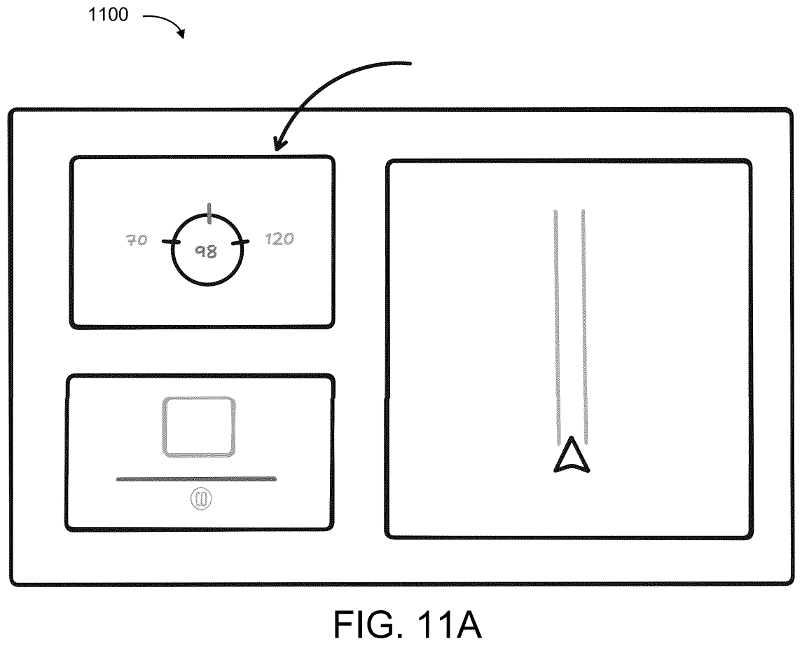

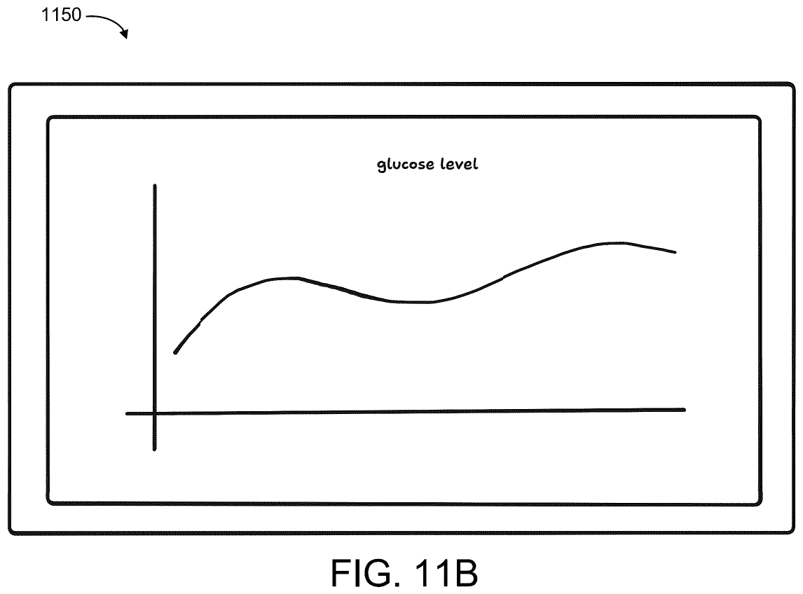

FIG. 11A illustrates an example interface 1100 depicting system components with sensor data displayed on a car dashboard with different levels of alerts. The interface is divided into sections, with the left portion showing the current glucose level (98 mg/dL) displayed in a circular gauge with threshold indicators, and a media control interface below. The right portion displays a navigation map showing the current route and vehicle position.

This dashboard integration represents the technical solution for real-time driver monitoring described throughout the patent. The implementation leverages vehicle connectivity capabilities through integration with modern infotainment systems such as Apple CarPlay and Android Auto, allowing the monitoring system 100 to display critical information directly on the vehicle's built−in screens. The system receives continuous glucose data from sensors 106 through secure API connections to diabetes management platforms, transmitting this information to the vehicle interface. The glucose display uses color-coding (green in this case) to indicate safe levels, with threshold indicators at 70 mg/dL and 120 mg/dL visually representing the boundaries of the safe range. The UI generator 160 employs responsive design principles to optimize the display for vehicle screens, ensuring critical information remains accessible regardless of the user platform utilized. This vehicle integration is particularly important for providing non-distracting, glanceable information that allows drivers to maintain awareness of their glucose levels without taking attention away from the road. The integration capability described here demonstrates the comprehensive approach to diabetes management during driving activities, creating a unified data stream that generates correlation analysis and pattern recognition.

FIG. 11B illustrates another example interface 1150 depicting system components with sensor data displayed on a car dashboard, specifically showing a time-series plot of glucose levels. This alternative display focuses on providing a trend view rather than a point−in-time reading, allowing the driver to understand not just the current glucose level but its directional movement and recent history.

The technical implementation of this dashboard visualization utilizes advanced data analytics to transform complex medical data into clear, actionable information that assists users in making informed decisions about diabetes management during driving activities. The system processes the continuous data stream from CGM devices, applying temporal filtering and statistical processing through the analysis module 126 to generate meaningful metrics and visualizations. The graphical representation is dynamically updated as new glucose readings are received, with the horizontal axis representing time and the vertical axis representing glucose levels. This implementation can integrate with vehicle systems through standardized protocols such as MirrorLink, Apple CarPlay, or Android Auto, which provide frameworks for mobile applications to display content on vehicle infotainment systems. The display adapts to different vehicle screen sizes and orientations through responsive design techniques implemented by the UI generator 160. The system intelligently adjusts the time scale of the visualization based on the duration of the current driving session, maintaining appropriate context for the displayed glucose trend. This visualization directly addresses the technical problems described herein by providing a clear, non-distracting way for drivers to monitor glucose trends, helping prevent situations where users might drive in a glucose-impaired state.

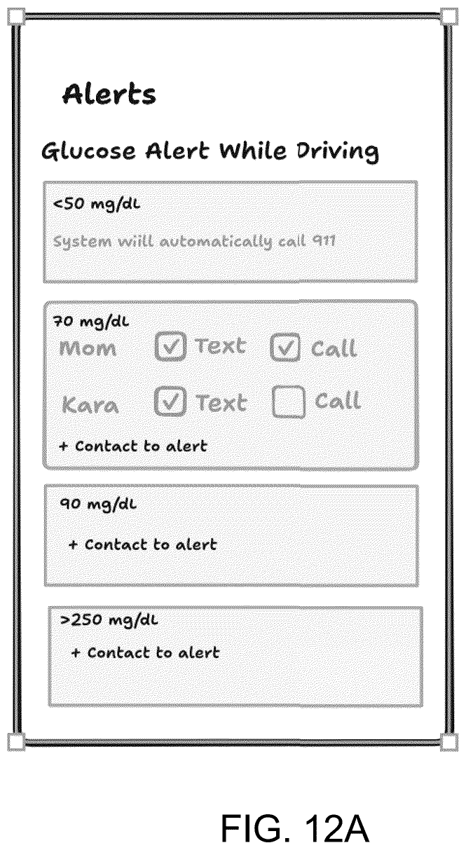

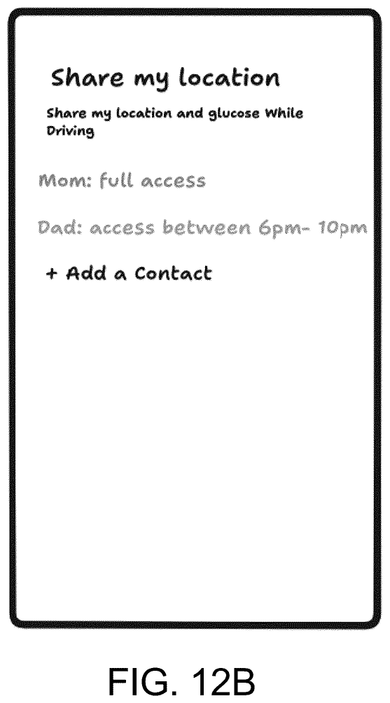

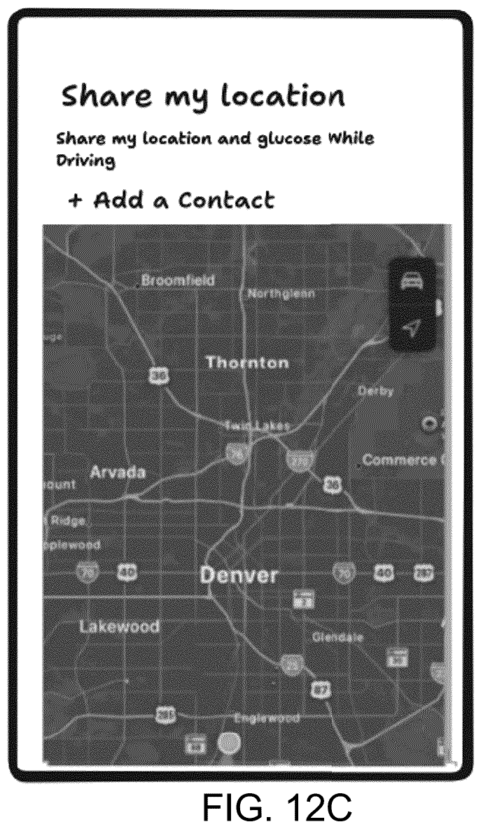

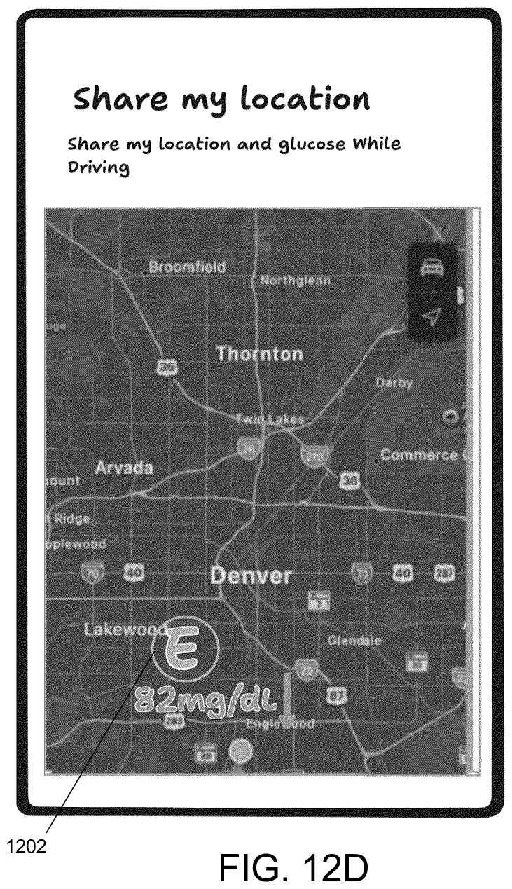

FIGS. 12A-12D illustrate an example of sharing features where a user can share live trip and blood sugar data with trusted family members for safety concerns. FIG. 12A illustrates an example user interface of a diabetes management application displaying an alert configuration screen. The interface allows the user to designate specific contacts to receive notifications when their blood glucose level falls below or exceeds a predefined range. The notification method may include an automatic text message or phone call triggered by the system.

FIG. 12B depicts the same user interface as shown in FIG. 12A but with an example configuration applied. In this instance, one contact has both text and call alerts enabled, while another contact is configured to receive only text notifications. The interface visually differentiates between the selected notification methods for each contact.

FIG. 12C illustrates an example user interface that enables users to share their real-time location along with their blood glucose data. The interface displays a map with the user's location and overlays relevant glucose readings, providing designated contacts with a combined view of spatial and health data for improved situational awareness.

FIG. 12D illustrates an example user interface similar to FIG. 12C but with additional controls 1202 that allow users to set limitations on when and how often their data is shared. The interface provides options for configuring time-based restrictions, frequency of updates, and specific conditions under which location and blood glucose data are shared with designated contacts. These settings enable users to maintain control over user-owned data visibility while ensuring timely alerts when needed.

### Methods

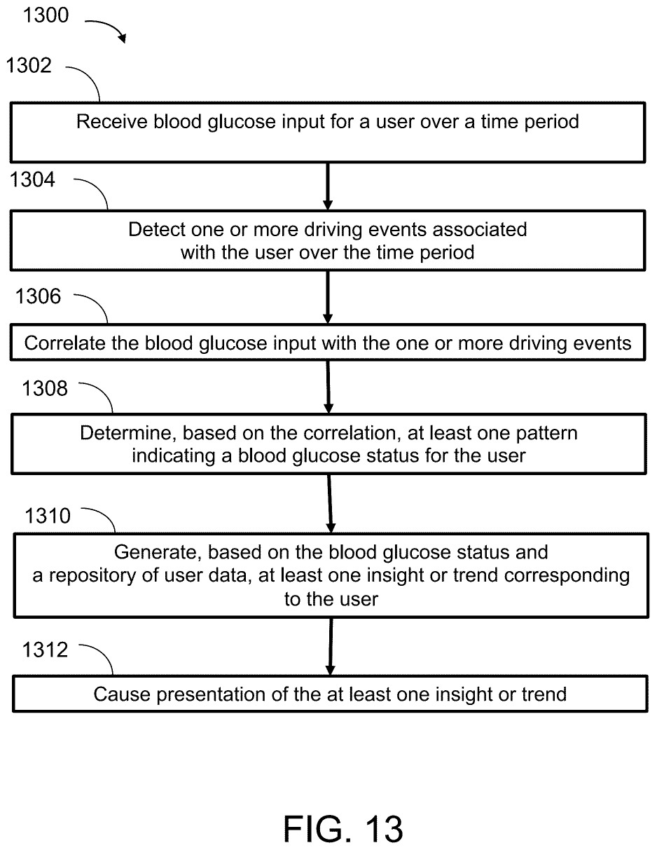

FIG. 13 illustrates a flow diagram of an example process 1300 for generating blood glucose insights correlated to driving-based events. The process 1300 which may function to assess blood glucose patterns when driving a vehicle. The process 1300 may additionally function to generate content, insights, and/or trends about the blood glucose levels and/or patterns determined while a user is driving a vehicle. In some embodiments, the process 1300 functions to generate insights to improve user health and habits. The process 1300 may be used for diabetes management], but can additionally or alternatively be used for any suitable applications, clinical or otherwise. The process 1300 can be configured and/or adapted to function for any suitable health-based analysis performed while a user is driving a vehicle.

At block 1302, the process 1300 includes receiving blood glucose input for a user over a time period. For example, the communication engine 144 of the monitoring system 100 may receive blood glucose input for the user. In some embodiments, the user may manually enter the blood glucose input one or more times over the time period. In some embodiments, the wearable device 108 may be worn by the user. Over a time period, the wearable device 108 may capture the blood glucose input and provide the input to the monitoring system 100.

At block 1304, the process 1300 includes detecting one or more driving events associated with the user over the time period. For example, the analysis module 126 may detect particular driving events over the time period. In particular, the analysis module 126 may function in combination with the risk assessment engine 134 to detect begin drive times, end drive times, risky health events associated with the blood glucose input or other sensed metric, risky driving events, or the like.

In some embodiments, the driving events may include a begin driving time and an end driving time and the blood glucose input may include a plurality of blood glucose measurements of the user captured historically and over the time period. For example, the analysis module 126 may detect a driving event beginning at 7:30 AM when the user's vehicle initiates movement as detected by GPS and accelerometer data that matches driving patterns (sustained speeds above 10 mph, movement along established roadways, etc.). The module may record this event with a timestamp and continue monitoring until the driving event concludes at 8:15 AM when the vehicle becomes stationary for more than 3 minutes and the engine is turned off. During this time period, the wearable device 108 or connected CGM system may have captured blood glucose readings at 5-minute intervals, recording values such as 127 mg/dL at driving start, 124 mg/dL at the 15-minute mark, 118 mg/dL at the 30-minute mark, and 112 mg/dL at driving conclusion, establishing a complete temporal dataset for analysis.

At block 1306, the process 1300 includes correlating the blood glucose input with the one or more driving events. For example, the pattern/trend determination engine 128 may perform this correlation by temporally mapping the glucose readings to specific driving event timestamps. The engine creates data pairs associating each glucose reading with its corresponding driving context, such as “pre-drive” (within 30 minutes before driving begins), “early-drive” (first third of drive duration), “mid-drive” (middle third), “late-drive” (final third), and “post-drive” (within 30 minutes after driving ends). This correlation process employs time-series alignment algorithms to accommodate irregular sampling intervals from different data sources, ensuring accurate temporal relationships between driving activities and glucose measurements. For instance, if a user's CGM records readings at 5-minute intervals but driving detection events have 1-minute granularity, the system applies interpolation techniques to establish precise correspondence between datasets, creating a unified timeline for subsequent pattern analysis.

This technical solution is necessarily rooted in computer technology because manual correlation of glucose readings with driving events across thousands of data points would be impractical and error-prone. A typical user generates 288 glucose readings per day (one every 5 minutes) and may have dozens of driving events per week, creating thousands of temporal data points that require precise alignment, interpolation, and phase assignment. Human practitioners cannot perform the cubic spline interpolation, weighted least squares regression, or modified Z-score outlier detection with the millisecond-level precision and consistency required for accurate pattern detection. The computational processing of asynchronous data streams with timestamp normalization and hysteresis-based phase assignment represents a solution to a problem that exists only in computer-implemented systems handling real-time sensor data.

In some embodiments, the correlating step of block 1306 is performed by executing a correlation algorithm comprising the following operations. First, the system retrieves a driving event record D comprising a start timestamp (T_start), an end timestamp (T_end), and optional metadata including route identifier, distance, and traffic conditions. Second, the system queries the blood glucose database to retrieve all glucose readings G[ ] where each reading g_i comprises a timestamp (t_i) and a glucose value (v_i) in mg/dL, filtering for readings where t_i falls within a correlation window defined as (T_start−30 minutes) to (T_end+30 minutes). Third, for each glucose reading g_i, the system assigns a temporal phase label by evaluating: (a) if t_i<T_start, assign “pre-drive” and calculate a pre-drive offset as (T_start−t_i) in minutes; (b) if T_start<=t_i<=T_end, calculate a normalized position P=(t_i-T_start)/(T_end-T_start) and assign “early-drive” if P<0.33, “mid-drive” if 0.33<=P<0.67, or “late-drive” if P>=0.67; (c) if t_i>T_end, assign “post-drive” and calculate a post-drive offset as (t_i−T_end) in minutes. Fourth, the system generates a correlated data structure C for the driving event comprising: the driving event record D, an array of phase-labeled glucose readings, computed metrics including starting glucose (the reading closest to T_start), ending glucose (the reading closest to T_end), minimum glucose during drive, maximum glucose during drive, glucose delta (ending minus starting), and rate of change calculated as glucose delta divided by drive duration in minutes. Fifth, the system applies linear interpolation to estimate glucose values at exact driving event boundaries when no reading exists precisely at T_start or T_end, using the formula: interpolated_value=v_a+((v_b−v_a)*(t_target−t_a)/(t_b−t_a)), where v_a and v_b are glucose values at timestamps t_a and t_b that bracket the target timestamp t_target. The resulting correlated data structure C is stored in the repository and made available for pattern analysis by subsequent processing modules.

In some embodiments, the system 100 implements a multi-step temporal alignment process to correlate asynchronous data streams from glucose monitoring devices and driving detection systems. For example, in a first step, the system 100 normalizes timestamps to UTC with millisecond precision using the formula T_normalized=T_local+UTC offset+leap_second_correction to ensure temporal consistency across different time zones and accounting for leap second adjustments. When glucose reading timestamps do not align exactly with driving event boundaries, the system applies cubic spline interpolation according to the formula G_interpolated(t)=a₃(t−t_i)³+a₂(t−t_i)²+a₁(t−t_i)+a₀, where coefficients a₀ through a₃ are computed from surrounding glucose readings using natural boundary conditions to provide smooth interpolation between measured values.

The system 100 then assigns each glucose reading to a driving phase using hysteresis to prevent rapid phase transitions that could introduce noise into the analysis. If a reading was previously classified as “pre-drive” and the time difference (t-T_start) exceeds negative 2 minutes, the system 100 transitions the reading to “early-drive” classification. Similarly, if previously in “early-drive” and the normalized position exceeds 0.35, the system transitions to “mid-drive,” with similar hysteresis applied to all phase boundaries using a +0.02 normalized position buffer to prevent oscillation between phases.

For rate-of-change calculation, the system 100 computes glucose rate-of-change using weighted least squares regression over a sliding 15-minute window according to the formula ROC(t)=(Σw_i·t_i·G_i·Σw_i·t_i·ΣG_i)/(Σw_i·t_i²−(Σw_i·t_i)²), where weights w_i decrease exponentially for older readings following the relationship w_i=exp(−λ·(t−t_i)) to give greater influence to more recent measurements. To handle measurement anomalies, the system applies modified Z-score outlier detection using the formula M_i=0.6745 (G_i−median (G))/MAD, where MAD equals the median of the absolute deviations from the median glucose value, specifically median (|G_i−median (G)|). Readings with an absolute modified Z-score exceeding 3.5 are flagged as outliers and replaced with interpolated values to prevent erroneous data from corrupting the analysis. This correlation algorithm produces a unified temporal dataset suitable for pattern analysis while handling real-world data irregularities such as sensor dropouts, timestamp misalignments, and measurement noise that commonly occur in continuous glucose monitoring systems.

The system 100 may detect driving events using data from multiple sensors available on mobile computing devices. In some embodiments, driving detection employs fusion of: Global Positioning System (GPS) data which provides location, speed, and heading information, accelerometer data which provides motion characteristics including vibration patterns and acceleration events, and gyroscope data (optional) which provides rotational information for enhanced activity classification

In some embodiments a driving event is detected when sustained speed exceeds a threshold value for a minimum duration or when the speed threshold is be between approximately 10 and approximately 25 miles per hour (approximately 15 to 40 kilometers per hour), and/or the minimum duration is between approximately 20 and approximately 60 seconds.

A minimum travel distance may be required, such as approximately 0.25 to approximately 1 mile. In some embodiments, driving is detected when speed exceeds approximately 15 miles per hour (approximately 24 kilometers per hour) for a sustained period of at least approximately 30 seconds. Hysteresis may be applied to prevent rapid state transitions. Once driving is detected, the driving state may persist until speed drops below a lower threshold (such as approximately 5 to approximately 10 miles per hour) for a duration (such as approximately 60 to approximately 120 seconds).

In some embodiments, accelerometer data is processed to classify transportation mode and distinguish driving from other activities such as walking, cycling, or public transit. Example features extracted from accelerometer data may include: Signal Vector Magnitude (SVM): Calculated as \sqrt{(ax²+ay²+az²)} where ax, ay, and az are acceleration components along three axes, differential SVM: Rate of change of SVM over time, and/or Hjorth parameters: Including activity (signal variance), mobility (ratio of variance of first derivative to signal variance), and complexity (ratio of mobility of first derivative to mobility of signal) Accelerometer data may be sampled at rates between approximately 25 Hz and approximately 100 Hz, with approximately 50 Hz being suitable for transportation mode classification. Classification may employ machine learning models such as Random Forest, Support Vector Machine, or neural network classifiers trained on labeled transportation mode data.

In some embodiments, driving detection is performed or supplemented by direct communication with vehicle systems. Integration approaches may include: Bluetooth connection to vehicle infotainment systems: Vehicle connectivity status indicates driving context, Android Automotive OS APIs: Including CarDrivingStateManager (providing Parked, Idling, Moving states) and CarPropertyManager (providing gear selection, vehicle speed), and OBD-II (On-Board Diagnostics) interface: Enabling access to vehicle speed, engine status, and other parameters via standardized diagnostic protocols.

At block 1308, the process 1300 includes determining, based on the correlation, at least one pattern indicating a blood glucose status, as explained in claim 5, for the user. For example, the pattern/trend determination engine 128 employs statistical analysis and machine learning techniques to identify recurring patterns. The engine analyzes the correlated data using time-series pattern recognition algorithms that can detect rate-of-change signatures, recurring temporal patterns, and statistically significant deviations during driving events compared to baseline periods. For instance, the system might determine that a particular user consistently experiences a 15-20 mg/dL drop in glucose levels during morning commutes lasting longer than 25 minutes, but maintains stable levels during evening drives of similar duration. Additional identified patterns might include glucose instability during stop-and-go traffic conditions compared to highway driving, or correlations between pre-drive glucose levels below 140 mg/dL and subsequent hypoglycemic risk during drives exceeding 45 minutes. These patterns are quantified with confidence intervals to assess their statistical significance and reliability. In some embodiments, the at least one pattern includes at least one of: a temporal pattern, a threshold violation pattern, a rate-of-change pattern, or a driving-correlated pattern, as described elsewhere herein.

The pattern determination process is necessarily rooted in computer technology because it requires simultaneous evaluation of multiple pattern types such as temporal patterns (e.g., post-meal glucose rises and dawn phenomenon), threshold violation patterns (hypoglycemic and hyperglycemic episodes), rate-of-change patterns (e.g., rapid decline or rapid rise), and driving-correlated patterns (e.g., pre-drive hypoglycemia tendency, driving-associated glucose drops) across weeks or months of historical data. The system 100 applies sliding window analysis with multiple window sizes, template matching against known pattern signatures, and anomaly detection to identify deviations from expected glucose behavior. For a single user with 30 days of driving history, this may involve analyzing over 8,000 glucose readings correlated with dozens of driving events, computing statistical significance using confidence intervals, and evaluating pattern persistence scores. This computational workload, combined with the requirement for consistent application of complex statistical methods and real-time processing as new data arrives, represents analytical capabilities that are achievable only through computer implementation.

At block 1310, the process 1300 includes generating, based on the blood glucose status and a repository of user data, at least one insight or trend corresponding to the user. For example, the insight generation engine 124 processes the identified patterns alongside historical user data from the repository to generate personalized insights. In some embodiments, the at least one insight or trend may include one or more of: blood glucose data over the time period, GPS instructions, and one or more links to access pattern-related content. For example, if the pattern shows consistent glucose drops during morning commutes, the system might generate an insight stating: “Morning Drive Alert: Your glucose typically drops by 18 mg/dL during weekday morning commutes over 25 minutes. Consider adjusting your breakfast carbohydrate intake or pre-drive insulin dosing.” This insight would be accompanied by a visualization of blood glucose trends during recent morning drives, GPS-based recommendations for potential stopping points along commonly traveled routes where food is available if needed, and links to educational content about optimizing morning glucose management for drivers with diabetes. The content/link generator 130 specifically tailors these resources to match the user's detected patterns and preferences stored in their profile data 103.

At block 1312, the process 1300 includes causing presentation of the at least one insight or trend. For example, the UI generator 160 transforms the insights into user-appropriate visualizations and interfaces for presentation through the user's preferred devices. For example, the presentation of the at least one insight or trend may include presenting an alert indication and a recommendation for accessing nearby food options when the blood glucose status is below a predefined safe blood glucose threshold and requesting additional blood glucose input at predefined intervals, in response to detecting that the recommendation was carried out. The UI generator 160 may deliver this content through multiple channels based on context: displaying non-urgent insights through the mobile app dashboard when the user is not driving, providing voice-enabled alerts through vehicle audio systems during active driving when glucose levels approach thresholds (e.g., “Your glucose is trending downward and currently at 78 mg/dL, approaching your safe driving threshold of 70 mg/dL”), and generating heads-up display notifications on compatible vehicle interfaces. When low glucose is detected, the communication engine 144 coordinates with mapping services to identify nearby food establishments matching the user's stored preferences and presents these options through navigation systems, while simultaneously scheduling follow-up glucose measurement prompts at 15-minute intervals after food consumption to verify recovery.

In some embodiments, the process 1300 may further include generating additional alerts in response to detecting that the recommendation was not carried out. For example, if a person did not acknowledge the first alert of mildly low blood sugar and blood sugar drops further, the user will be alerted with a navigation map to direct to safe places or food places where a person can pull over a car and treat low blood sugar. Again, if the user ignores second alerts and blood sugar drops further, the automatic call will be placed to a family member or whosoever is a designated person to make sure the driver is safe. In some embodiments, an additional alert may include accessing and dialing or otherwise contacting or alerting one or more predefined emergency contacts.

In some embodiments, the process 1300 may further include generating additional alerts in response to detecting that the recommendation carried out was ineffective at raising the blood glucose status above the predefined safe blood glucose threshold. For example, an additional alert may include accessing and dialing or otherwise contacting or alerting one or more predefined emergency contacts.

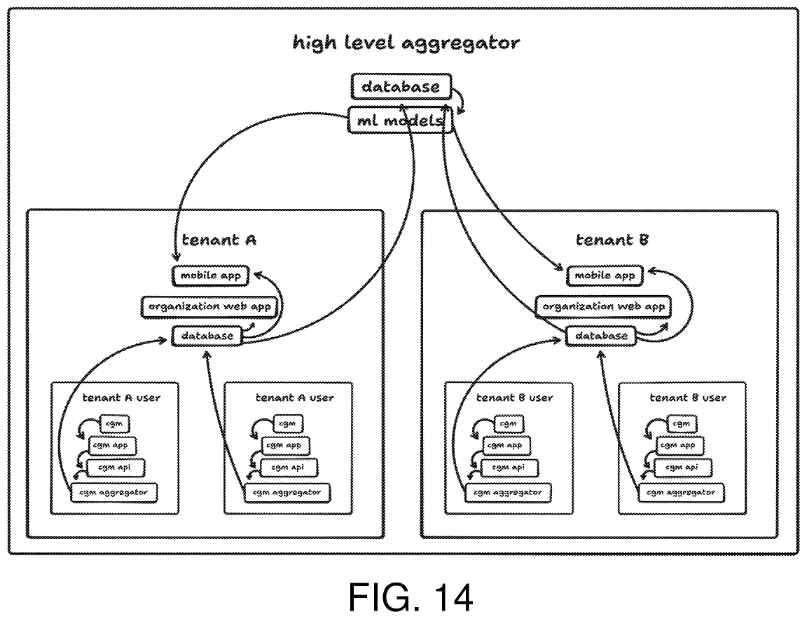

FIG. 14 illustrates a multi-tenant commercial architecture 1400 for enterprise deployment of the monitoring system 100. In some embodiments, the system may be deployed to serve multiple organizational tenants 1402, 1404, such as fleet operators, insurance companies, healthcare organizations, or employers with employees who drive as part of their work responsibilities. Each tenant may include a tenant-specific mobile app 1406 for individual users, an organization web app 1408 providing administrative dashboards for monitoring driver glucose and safety metrics, generating compliance and risk reports, and managing user onboarding and permissions, and a tenant database 1410 that maintains data isolation between organizations. Individual users within each tenant may connect their CGM devices through a CGM app and CGM API to a CGM aggregator 1412 that collects and normalizes glucose data before transmission to the tenant database. A high-level aggregator 1414 may include a centralized database 1416 and shared ML models 1418 that receive anonymized, aggregated data from tenant databases to enable federated learning across the platform. This architecture allows the ML models 162 to improve prediction accuracy by learning from diverse driving and glucose patterns across multiple organizations while the data validation module 138 ensures that individual user data remains isolated within tenant boundaries, maintaining HIPAA compliance and organizational data sovereignty. The shared ML models may provide enhanced predictive capabilities that benefit all tenants without compromising the privacy of any individual organization's data.

The data validation module 138, illustrated in FIG. 1, implements multi-layered security protocols to protect sensitive user health data while ensuring data integrity. These protocols include end-to-end AES-256 encryption for all glucose and driving data transmissions, with rotating encryption keys generated using NIST-compliant cryptographic algorithms. The module employs OAuth 2.0 with multi-factor authentication for API integrations with external devices and platforms, while implementing tokenization to prevent storage of personally identifiable information within the same database as health metrics. Additionally, the system applies differential privacy techniques that add calibrated noise to aggregated datasets before analysis, mathematically guaranteeing individual privacy while preserving statistical utility. All data access is governed by role-based permissions with comprehensive audit logging that captures timestamp, user identity, accessed data fields, and the specific actions performed. These security measures collectively ensure HIPAA compliance while maintaining data quality for accurate insight generation.

The monitoring system 100 may generate output 170 including, but not limited to alerts (and voice enable alerts), estimated glucose values, events, insights, trends, educational content, community engagement features, trip sharing features and the like. For example, trip starting and ending blood sugar value with trends of blood sugar during trips, pattern of high or low blood sugar while driving and common solutions to prevent low blood sugars. These patterns or features could be shared with health care providers or family members.

In operation, the monitoring system 100 may support users with diabetes through a comprehensive, personalized approach as illustrated by the following example. Consider Jessica, a 35-year-old with Type 1 diabetes who commutes daily to work. Her morning begins when her CGM sensor 106 transmits a blood glucose reading of 142 mg/dL to the monitoring system 100, which simultaneously accesses her profile data 103 indicating her preferred safe glucose range (80-180 mg/dL) and historical patterns showing glucose typically drops during morning commutes. As she prepares to leave, the UI generator 160 displays her current glucose (138 mg/dL) alongside a prediction from the ML models 162 that her glucose may drop 20-30 mg/dL during her typical 35-minute commute. When Jessica begins driving, the analysis module 126 detects this driving event through GPS and motion sensor data, recording it as trip start 105 and triggering enhanced monitoring protocols through the event detector 148. Ten minutes into her drive, the data validation module 138 confirms a valid glucose reading of 125 mg/dL, while the driving analysis module 136 correlates this drop with the moderate traffic conditions. As her glucose continues declining to 112 mg/dL, the pattern/trend determination engine 128 identifies this as a faster-than-typical decline, prompting the predictive alert module 140 to calculate that she may approach her safety threshold before reaching her destination. The alert/message generator 146 prepares a first-level alert, which the delivery timing optimizer 152 strategically delivers via voice when Jessica stops at a red light: “Your glucose is currently 107 mg/dL and trending downward. Consider having a snack at your next opportunity.” When Jessica realizes she has no snack available, the safety recommendation generator 142 works with the content/link generator 130 to identify a nearby convenience store, and the UI generator 160 displays this reroute option. After Jessica purchases and consumes juice, logging this action through the feedback collection interface 154, the sensors 106 detect her glucose stabilizing at 90 mg/dL and beginning to rise. Upon arrival at her workplace with a glucose reading of 108 mg/dL, the system records the trip end 105 and stores the complete glucose curve for analysis. Later that day, the insight generation engine 124 processes this data, identifying that Jessica's glucose drops more rapidly during congested traffic compared to flowing traffic. The content/link generator 130 then identifies relevant educational content, which the ranking engine 158 prioritizes for presentation. When Jessica checks the app that evening, she receives personalized recommendations based on the newly identified pattern, thus completing the monitoring, intervention, and insight generation cycle that characterizes the system's operation.

The multi-tenant federated learning architecture solves a technical problem specific to computer networks: how to improve predictive models using distributed data while maintaining strict data isolation between organizations. This problem does not exist outside of computer-implemented systems because it requires simultaneous processing of data from multiple isolated databases, application of differential privacy techniques with calibrated noise injection, coordination of model training across distributed computing nodes, and secure transmission of anonymized gradients while preventing reconstruction of source data. The technical solution implements row-level security policies, tenant-specific encryption keys derived through HKDF-SHA256 with per-tenant salts, and automated partition management that creates and drops database partitions based on temporal criteria. These database-level and network-level isolation mechanisms address problems that are inherent to multi-tenant cloud computing architectures and have no analog in manual or paper-based diabetes management systems.

In some aspects, the techniques described herein relate to a computer-implemented method that is HIPAA compliant. In general, the systems described herein may generate requisite warnings to protect the information contained in data accessed and/or stored by such systems in compliance with the Health Insurance Portability and Accountability Act (HIPAA). Accordingly, the systems and methods described herein provide compliance with HIPAA, and other privacy requirements regarding patient medical data, or other personal information.

The systems and methods described herein and variations thereof can be embodied and/or implemented at least in part as a machine configured to receive a computer-readable medium storing computer-readable instructions. The instructions are preferably executed by computer-executable components preferably integrated with the system and one or more portions of the processor 164 on the system 100 and/or another computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A computer-implemented method for monitoring blood glucose of a user during operation of a vehicle, the method comprising:
receiving blood glucose input for a user over a time period;
detecting one or more driving events associated with the user over the time period;
correlating the blood glucose input with the one or more driving events;
determining, based on the correlation, at least one pattern indicating a blood glucose status for the user, wherein the at least one pattern comprises at least one of: a temporal pattern, a threshold violation pattern, a rate-of-change pattern, or a driving-correlated pattern;
generating, based on the blood glucose status and a repository of user data, at least one insight or trend corresponding to the user; and
causing presentation of the at least one insight or trend.

2. The computer-implemented method of claim 1, wherein the presentation comprises:
presenting an alert indication and a recommendation for accessing nearby food options when the blood glucose status is below a predefined safe blood glucose threshold; and
requesting additional blood glucose input at predefined intervals, in response to detecting that the recommendation was carried out.

3. The computer-implemented method of claim 2, wherein the at least one insight or trend further comprises one or more of:
blood glucose data over the time period;
GPS instructions; and
one or more links to access pattern-related content.

4. The computer-implemented method of claim 2, further comprising:
generating additional alerts in response to detecting that the recommendation was not carried out or detecting that the recommendation carried out was ineffective at raising the blood glucose status above the predefined safe blood glucose threshold; and
accessing one or more predefined emergency contacts.

5. The computer-implemented method of claim 1, wherein:
the driving events comprise a begin driving time and an end driving time; and
the blood glucose input comprises a plurality of blood glucose measurements of the user captured historically and over the time period.

6. The computer-implemented method of claim 1, further comprising:
predicting future blood glucose levels during a predicted driving duration over a prediction horizon ranging from about 15 minutes to about 90 minutes,
wherein the predicting is based on at least one of: historical glucose readings during prior driving events, a rate of change of current blood glucose levels, driving duration, time of day, meal information, insulin dosing information, or environmental factors.

7. The computer-implemented method of claim 1, wherein determining the at least one pattern indicating a blood glucose status for the user comprises:
calculating a rate of change of a blood glucose level based on a plurality of sequential blood glucose measurements for the user;
classifying the rate of change as one of: a moderate falling rate, a rapid falling rate, or a critical falling rate; and
generating an alert level based on the classified rate of change in combination with a current blood glucose value.

8. A system for analyzing blood glucose while driving a vehicle, comprising:
a computing unit comprising:
one or more sensors;
a processor; and
memory communicably coupled to the processor, wherein the memory stores processor-executable instructions, which when executed by the processor, cause the processor to:
receive blood glucose input for a user over a time period;
detect one or more driving events associated with the user over the time period;
correlate the blood glucose input with the one or more driving events;
determine, based on the correlation, at least one pattern indicating a blood glucose status for the user;
generate, based on the blood glucose status and a repository of user data, at least one insight or trend corresponding to the user; and
cause presentation of the at least one insight or trend.

9. The system of claim 8, wherein the processor-executable instructions, when executed by the processor, further cause the processor to:
expose an application programming interface (API) configured to:
receive blood glucose data from one or more third-party continuous glucose monitoring platforms;
transmit driving-correlated glucose insights to authorized external applications; and
authenticate API requests using token-based authentication with role-based access controls.

10. The system of claim 9, wherein the API is further configured to:
receive driving event data from vehicle telematics systems;
provide real-time glucose status updates to fleet management platforms; and
generate compliance reports for organizational administrators.

11. The system of claim 8, wherein the computing unit is integrated with at least one of: a vehicle infotainment system, a mobile computing device, or a wearable device.

12. The system of claim 8, wherein the processor executes machine learning models trained to predict glucose trajectories during the one or more driving events.

13. The system of claim 8, wherein the processor-executable instructions, when executed by the processor, further cause the processor to:
predict future blood glucose levels during a predicted driving duration over a prediction horizon ranging from about 15 minutes to about 90 minutes,
wherein the prediction is based on at least one of: historical glucose readings during prior driving events stored in the memory, a calculated rate of change of current blood glucose levels, predicted driving duration, time of day, meal logging data, insulin administration records, or traffic conditions.

14. The system of claim 8, wherein the processor-executable instructions, when executed by the processor, further cause the processor to:
calculate a rate of change of a blood glucose level based on a plurality of sequential blood glucose measurements for the user;
classify the rate of change as one of: a moderate falling rate, a rapid falling rate, or a critical falling rate; and
generate an alert level based on the classified rate of change in combination with a current blood glucose value.

15. A non-transitory computer-readable storage medium storing processor-executable instructions that, when executed by one or more processors, cause the one or more processors to perform operations comprising:
receiving blood glucose input for a user over a time period;
detecting one or more driving events associated with the user over the time period;
correlating the blood glucose input with the one or more driving events;
determining, based on the correlation, at least one pattern indicating a blood glucose status for the user, wherein the at least one pattern comprises at least one of: a temporal pattern, a threshold violation pattern, a rate-of-change pattern, or a driving-correlated pattern;
generating, based on the blood glucose status and a repository of user data, at least one insight or trend corresponding to the user; and
causing presentation of the at least one insight or trend.

16. The non-transitory computer-readable storage medium of claim 15, wherein the presentation comprises:
presenting an alert indication and a recommendation for accessing nearby food options when the blood glucose status is below a predefined safe blood glucose threshold; and
requesting additional blood glucose input at predefined intervals, in response to detecting that the recommendation was carried out.

17. The non-transitory computer-readable storage medium of claim 16, wherein the at least one insight or trend further comprises one or more of:
blood glucose data over the time period;
GPS instructions; and
one or more links to access pattern-related content.

18. The non-transitory computer-readable storage medium of claim 16, further comprising:
generating additional alerts in response to detecting that the recommendation was not carried out or detecting that the recommendation carried out was ineffective at raising the blood glucose status above the predefined safe blood glucose threshold; and
accessing one or more predefined emergency contacts.

19. The non-transitory computer-readable storage medium of claim 15, wherein:
the driving events comprise a begin driving time and an end driving time; and
the blood glucose input comprises a plurality of blood glucose measurements of the user captured historically and over the time period.

20. The non-transitory computer-readable storage medium of claim 15, further comprising:
predicting future blood glucose levels during a predicted driving duration over a prediction horizon ranging from about 15 minutes to about 90 minutes, wherein the predicting is based on at least one of: historical glucose readings during prior driving events, a rate of change of current blood glucose levels, driving duration, time of day, meal information, insulin dosing information, or environmental factors.

21. The non-transitory computer-readable storage medium of claim 15, wherein determining the at least one pattern indicating a blood glucose status for the user comprises:
calculating a rate of change of a blood glucose level based on a plurality of sequential blood glucose measurements for the user;
classifying the rate of change as one of: a moderate falling rate, a rapid falling rate, or a critical falling rate; and
generating an alert level based on the classified rate of change in combination with a current blood glucose value.

22. A computer-implemented method for providing a multi-tenant blood glucose monitoring service, the method comprising:
provisioning a tenant account for an organization within a multi-tenant platform;
receiving, via an organization web application, configuration parameters comprising alert thresholds, user permissions, and reporting preferences for the tenant account;
enrolling one or more users associated with the organization, wherein each user is associated with a continuous glucose monitoring device;
receiving blood glucose data from the one or more users via tenant-specific mobile applications;
storing the blood glucose data in a tenant-isolated database that maintains data separation between organizations;
correlating the blood glucose data with driving events detected for each user;
generating organizational reports comprising aggregated glucose metrics, driving safety scores, and compliance indicators; and
presenting the organizational reports via the organization web application.

23. The computer-implemented method of claim 22, further comprising:
transmitting anonymized glucose and driving pattern data from the tenant-isolated database to a federated learning aggregator;
training shared machine learning models using the anonymized data from a plurality of tenant databases; and
distributing improved predictive models to the plurality of tenant databases, wherein individual user data remains isolated within respective tenant boundaries.

24. The computer-implemented method of claim 22, wherein the organization comprises one or more of:
a fleet operator managing commercial drivers;
an insurance company monitoring policyholder driving behavior;
a healthcare organization coordinating patient care; or
an employer with employees who drive as part of work responsibilities.

25. The computer-implemented method of claim 22, further comprising:
managing subscription access to the multi-tenant platform based on a service tier associated with the tenant account;
enforcing usage limits corresponding to the service tier; and
providing tiered access to features comprising real-time monitoring, historical analytics, API access, and custom reporting based on the service tier.

26. The computer-implemented method of claim 1, wherein the method is performed to:
manage a commercial driver;
monitor driving behavior of an insurance policyholder;
monitor healthcare coordinator behavior; or
monitor employee driving.
